SEQUENCE LISTING (26 sequences)

SEQ ID NO: 1 - DNA, 1668 bp, Zea mays
  misc_feature (603)..(603): n is a, c, g, or t
atgggttccgtagacgcggcgatcgcggtgccggtgccggcggcggaggagaaggcggtggaggagaaggcgatggtgttccggtccaagcttcccgacatcgagatcgacagcagcatggcgctgcacacctactgcttcgggaagatgggcgaggtggcggagcgggcgtgcctgatcgacgggctgacgggcgcgtcgtacacgtacgcggaggtggagtccctgtcccggcgcgccgcgtcggggctgcgcgccatgggggtgggcaagggcgacgtggtgatgagcctgctccgcaactgccccgagttcgccttcaccttcctgggcgccgcccgcctgggcgccgccaccaccacggccaacccgttctacaccccgcacgaggtgcaccgccaggcggaggcggccggcgcccggctcatcgtgaccgaggcctgcgccgtggagaaggtgcgggagttcgcggcggagcggggcatccccgtggtcaccgtcgacgggcgcttcgacggctgcgtggagttcgccgagctgatcgcggccgaggagctggaggctgacgccgacatccaccccgacgacgtcgtcgcgctgccntactcctccggcaccaccgggctgcccaagggcgtcatgctcacccaccgcagcctcatcaccagcgtcgcgcagcaggttgatggcgagaacccgaacctgtacttccgcaaggacgacgtggtgctgtgcctgctgccgctgttccacatctactcgctgaactcggtgctgctggccggcctgcgcgcgggctccaccatcgtgatcatgcgcaagttcgacctgggcgcgctggttgacctggtgcgcaggtacgtgatcaccatcgcgcccttcgtgccgcccatcgtggtggagatcgccaagagcccccgcgtgaccgccggcgacctcgcgtccatccgcatggtcatgtccggcgccgcgcccatgggcaaggagctccaggacgccttcatggccaagatccccaatgccgtgctcgggcaggggtacgggatgacggaggcaggccccgtgctggcgatgtgcctggccttcgccaaggagccgtacccggtcaagtccgggtcgtgcggcaccgtggtgcggaacgcggagctgaagatcgtcgaccccgacaccggcgccgccctcggccggaaccagcccggcgagatctgcatccgcggggagcagatcatgaaaggttacctgaacgaccccgagtcgacgaagaacaccatcgaccaggacggctggctgcacaccggcgacatcggctacgtggacgacgacgacgagatcttcatcgtcgacaggctcaaggagatcatcaagtacaagggcttccaggtgccgccggcggagctggaggcgctcctcatcacgcacccggagatcaaggacgccgccgtcgtctcaatgaacgacgaccttgctggtgaaatcccggtcgccttcatcgtgcggaccgaaggttctcaagtcaccgaggatgagatcaagcaattcgtcgccaaggaggtggttttctacaagaagatccacaaggtcttcttcaccgaatccatccccaagaacccgtcgggcaagatcctgaggaaggacttgagagccaggctcgccgccggtgttcactga

SEQ ID NO: 2 - protein, 555 aa, Zea mays
MGSVDAAIAVPVPAAEEKAVEEKAMVFRSKLPDIEIDSSMALHTYCFGKMGEVAERACLIDGLTGASYTYAEVESLSRRAASGLRAMGVGKGDVVMSLLRNCPEFAFTFLGAARLGAATTTANPFYTPHEVHRQAEAAGARLIVTEACAVEKVREFAAERGIPVVTVDGRFDGCVEFAELIAAEELEADADIHPDDVVALPYSSGTTGLPKGVMLTHRSLITSVAQQVDGENPNLYFRKDDVVLCLLPLFHIYSLNSVLLAGLRAGSTIVIMRKFDLGALVDLVRRYVITIAPFVPPIVVEIAKSPRVTAGDLASIRMVMSGAAPMGKELQDAFMAKIPNAVLGQGYGMTEAGPVLAMCLAFAKEPYPVKSGSCGTVVRNAELKIVDPDTGAALGRNQPGEICIRGEQIMKGYLNDPESTKNTIDQDGWLHTGDIGYVDDDDEIFIVDRLKEIIKYKGFQVPPAELEALLITHPEIKDAAVVSMNDDLAGEIPVAFIVRTEGSQVTEDEIKQFVAKEVVFYKKIHKVFFTESIPKNPSGKILRKDLRARLAAGVH

SEQ ID NO: 3 - DNA, 1468 bp, Zea mays
ggcacgagctcggctgctacgtagctatatatagctgcagaacagttttagttaccggactgtaggcacaccaagctagatatccaaggggcccccccggaaactagctagctaggatgccaacagcagaggcgacgacgcccgtgccgccggagctgtcctccgggcaagggcggacggtttgcgtgaccggagctggagggttcattgcctcctggctcgtcaagcgcctcctggagaagggctacacagtccgcggcacggtcaggaaccctgtggatccaaagaacgaccacctgagggcccttgacggcgccgtcgaccgtctcgtcctcctccgcgccgacctgctggacccacagagccttgccgaggccttctccggctgtgacggcgtcttccacgccgcctccccggtcaccgatgaccctgagatgatgatcgagccagcgatccggggcacgcgctacgtgatggcggcggcggacgacgccggagtgaagcgcgtcgtgttcacgtcctccatcggcaccgtgtacatgaacccctaccgtgaccccagcaagcctgtggacgacacctgctggagcgacctcgagtattgcaagaacacccagaactggtactgctacgccaagacggtggcggagcagggcgcgtgggaggtggcgcggaagcgagggctggacctggtggtggtgaacccggtgctggtgctgggcccgctgctgcagccgacggtgaacgccagcacggaccacgtgatgaagtacctgacggggtcggccacgacctacgtgaacgcggcgcaggcgtacgtgcacgtcagggacgtggccgaggcgcacgtccgggtgtacgaggcgccccacgcgcacggccgctacatctgcgccgagagcaccctgcaccgcggcgacctctgccgcgtcctcgccaagctcttcccggagtaccccgtacccaccaagtgcaaggaccaggttaaccctccggtggtaggatacaagttcaccaaccagaggctcaaggatctggggatggacttcgtgccggtcctgcagtgcctctacgagacggtcaccagcctccaggagaaaggcatgctgcccgtgcttccgacaaaacaagaccatgaccaactcggcaaatcatgatcatattcatatccctcgttgacgatactagctgctgctaccacgcatatcatatcatatatataagatatatatatatatagtggattcatatagttaaccttatatatatgtgtgtgtgtgtgtgttattgtgtgttattagtgtgtacaattaaatggcaactctaatgggtgcatgcatctgcatgcatgctttcctgacaagtgccacacatgcatgcaatgtatctctttggacgatttatatacaatactccgttgccaaataaaaaaaaaaaaaaaaaaaaaaaaaaaaaaaaaaaaaaaaaa

SEQ ID NO: 4 - protein, 346 aa, Zea mays
MPTAEATTPVPPELSSGQGRTVCVTGAGGFIASWLVKRLLEKGYTVRGTVRNPVDPKNDHLRALDGAVDRLVLLRADLLDPQSLAEAFSGCDGVFHAASPVTDDPEMMIEPAIRGTRYVMAAADDAGVKRVVFTSSIGTVYMNPYRDPSKPVDDTCWSDLEYCKNTQNWYCYAKTVAEQGAWEVARKRGLDLVVVNPVLVLGPLLQPTVNASTDHVMKYLTGSATTYVNAAQAYVHVRDVAEAHVRVYEAPHAHGRYICAESTLHRGDLCRVLAKLFPEYPVPTKCKDQVNPPVVGYKFTNQRLKDLGMDFVPVLQCLYETVTSLQEKGMLPVLPTKQDHDQLGKS

SEQ ID NO: 5 - DNA, 1398 bp, Zea mays
ggcacgagaggacacaagcgagcgctagccagaagagcagctgcaggtactattatcatcgtcgtcgtcgtcgccaggatgaccgtcgtcgacgccgtcgtctcctccaccgatgccggcgcccctgccgccgccgccgcaccggtaccggcggggaacgggcagaccgtgtgcgtcaccggcgcggccgggtacatcgcctcgtggttggtgaagctgctgctcgagaagggatacactgtgaagggcaccgtgaggaacccagatgacccgaagaacgcgcacctcagggcgctggacggcgccgccgagcggctgatcctctgcaaggccgatctgctggactacgacgccatctgccgcgccgtgcagggctgccagggcgtcttccacaccgcctcccccgtcaccgacgacccggagcaaatggtggagccggcggtgcgcggcaccgagtacgtgatcaacgcggcggcggaggccggcacggtgcggcgggtggtgttcacgtcgtccatcggcgccgtgaccatggaccccaagcgcgggcccgacgtcgtggtcgacgagtcgtgctggagcgacctcgagttctgcgagaaaaccaggaactggtactgctacggcaaggcggtggcggagcaggcggcgtgggaggcggcccggcggcggggcgtggacctggtggtggtgaaccccgtgctggtggtgggccccctgctgcaggcgacggtgaacgccagcatcgcgcacatcctcaagtacctggacggctcggcccgcaccttcgccaacgccgtgcaggcgtacgtggacgtgcgcgacgtggccgacgcgcacctccgcgtcttcgagagcccccgcgcgtccggccgccacctctgcgccgagcgcgtcctccaccgcgaggacgtcgtccgcatcctcgccaagctcttccccgagtaccccgtcccagccaggtgctccgacgaggtgaatccgcggaagcagccgtacaagttctccaaccagaagctccgggacctggggctgcagttccggccggtcagccagtcgctttacgacacggtgaagaacctccaggagaagggccacctgccggtgctcggagagcggacgacgacggaggccgccgacaaggatgcccccgcggccgagatgcagcagggagggatcgccatccgtgcctgagagggcgatgccacacatgaacacaaagcaatgttcatactgctgccctgcacctgcaccttcccctgctgtgtaaacaggcctgtgtttgttctggctgatagtgatgtaccctaagacttgtaacgtcatgttcgttcttgtgaactatagcgagtgaataaaattggttaatgttggatattcaaaaaaaaaaaaaaaaaa

SEQ ID NO: 6 - protein, 371 aa, Zea mays
MTVVDAVVSSTDAGAPAAAAAPVPAGNGQTVCVTGAAGYIASWLVKLLLEKGYTVKGTVRNPDDPKNAHLRALDGAAERLILCKADLLDYDAICRAVQGCQGVFHTASPVTDDPEQMVEPAVRGTEYVINAAAEAGTVRRVVFTSSIGAVTMDPKRGPDVVVDESCWSDLEFCEKTRNWYCYGKAVAEQAAWEAARRRGVDLVVVNPVLVVGPLLQATVNASIAHILKYLDGSARTFANAVQAYVDVRDVADAHLRVFESPRASGRHLCAERVLHREDVVRILAKLFPEYPVPARCSDEVNPRKQPYKFSNQKLRDLGLQFRPVSQSLYDTVKNLQEKGHLPVLGERTTTEAADKDAPAAEMQQGGIAIRA

SEQ ID NO: 7 - DNA, 1481 bp, Zea mays
ctcgatcgtcgtcatcacgctcgaccgcacaactgcaccaaggggggaggagacctaaaaactactacatcttttagctacacatctagctaaagatcgagaggggtaaataaggacgagcgggcgcgagctagaagagcagctgcaggtactaccatcatcgtcgtcgtcgtcgccaggatgaccgtcgtcgacgccgtcgtctcctccaccgatgccggcgcccctgctgccgccgccaccgcggtaccggcggggaacgggcagaccgtgtgcgtgaccggcgcggccgggtacatcgcctcgtggttggtgaagctgctgctcgagaagggatacactgtgaagggcaccgtcaggaacccagatgacccgaagaacgcgcacctcaaggcgctggacggcgccgccgagcggctgatcctctgcaaggccgatctgctggactacgacgccatctgccgcgccgtgcagggctgccagggcgtcttccacaccgcctcccccgtcaccgacgacccggagcaaatggtggagccggcggtgcgcggcaccgagtacgtgatcaacgcggcggcggaggccggcacggtgcggcgggtggtgttcacgtcgtccatcggcgccgtgaccatggaccccaagcgcgggcccgacgtcgtggtcgacgagtcgtgctggagcgacctcgagttctgcgagaaaaccaggaactggtactgctacggcaaggcggtggcggagcacgcggcgtgggagacggcccggcggcggggcgtggacctggtggtggtgaaccccgtgctggtggtgggccccctgctgcaggcgacggtgaacgccagcatcgcgcacatcctcaagtacctggacggctcggcccgcaccttcgccaacgccgtgcaggcgtacgtggacgtgcgcgacgtggccgacgcgcacctccgcgtcttcgagagcccccgcgcgtccggccgccacctctgcgccgagcgcgtcctccaccgcgaggacgtcgtccgcatcctcgccaagctcttccccgagtaccccgtcccagccaggtgctccgacgaggtgaatccgcggaagcagccgtacaagttctccaaccagaagctccgggacctggggctgcagttccggccggtcagccagtcgctttacgacacggtgaagaacctccaggagaagggacacctgccggtgctcggagagcggacgacgacggaggccgccgacaaggatgcccccacggccgagatgcagcagggagggatcgccatccgtgcctgagagggcgatgccacacatgaacacaaagcaatgttcatactgctgccctgcacctgctgtgtaaacaggcctgtgtttgttctggctgatagtgatgtaccctaagacttgtaacgtcatgttcgttcttgtgaactatagcgagtgaataaaattggttaatgttggatgttcaaaaaaaaaaa

SEQ ID NO: 8 - protein, 371 aa, Zea mays
MTVVDAVVSSTDAGAPAAAATAVPAGNGQTVCVTGAAGYIASWLVKLLLEKGYTVKGTVRNPDDPKNAHLKALDGAAERLILCKADLLDYDAICRAVQGCQGVFHTASPVTDDPEQMVEPAVRGTEYVINAAAEAGTVRRVVFTSSIGAVTMDPKRGPDVVVDESCWSDLEFCEKTRNWYCYGKAVAEHAAWETARRRGVDLVVVNPVLVVGPLLQATVNASIAHILKYLDGSARTFANAVQAYVDVRDVADAHLRVFESPRASGRHLCAERVLHREDVVRILAKLFPEYPVPARCSDEVNPRKQPYKFSNQKLRDLGLQFRPVSQSLYDTVKNLQEKGHLPVLGERTTTEAADKDAPTAEMQQGGIAIRA

SEQ ID NO: 9 - DNA, 1516 bp, Zea mays
ccggcgctcgcgcggctttctttcccaactccgacgaaggctagctacaccaccttgtgcgggctcgtctccatcgcccgccacccgctccgtcgtcgtcgtccccgccgcgccgatcccgaatcgaatggggagcctggcgtccgagaggaaggtggtcgggtgggccgccagggacgccaccggacacctctccccctactcctacaccctcaggaacacaggccctgaagatgtggtggtgaaggtgctctactgcgggatctgccacacggacatccaccaggccaagaaccacctcggggcttcaaagtatcctatggtccctgggcacgaggtggtcggcgaggtggtggaggtcgggcccgaagtggccaagtacggcgtcggcgacgtggtaggcgtcggggtgatcgttgggtgctgccgcgagtgcagcccctgcaaggccaacgttgagcagtactgcaacaagaagatctggtcatacaacgacgtctacactgatggacggcccacgcagggtggattcgcctccaccatggtcgtcgaccagaagtttgtggtgaagatcccggcgggtctggctccggagcaagcggcgccgctgctgtgcgctggcgtgacggtgtacagcccgctgaagcactttgggctgacgaacccgggcctccgtggcggcatcctgggcctcggcggcgtgggccacatgggcgtgaaggtagccaaggccatgggccaccacgtgacggtgatcagctcgtcgtccaagaagcgcgcggaggcaatggaccacctcggcgcggacgcgtacctagtgagctcggacgccgcggccatggcggcggccgccgactcgctggactacatcatcgacacggtgcccgtgcaccacccgctggagccgtacctggcgctgctgaagctggacggcaagctcgtgctgctgggcgtcatcggcgagcccctgagcttcgtgtcgcccatggtgatgctggggcggaaggccatcacggggagcttcatcggcagcatcgacgagaccgctgaggtgcttcagttctgcgtcgacaaggggctcacctcccagatcgaggtggtcaagatggggtacgtgaacgaggcgctggagcggctggagcgcaacgacgtccgctaccgcttcgtcgtcgacgtcgccggtagcaacgtcgaggcggaggcggcggcggcggatgcggccagcaactgatggcaccgcgtcgtcgagtcgaaccacgtctgtgcgccgcgtgcaacgttcgttcgggtcgagtctgcgtgcaacgttctgcttcctttactagttgttgtctttccgccttcttgccgttctgttctgggctttgagatgagacgatggatggtcagtttttaatgtcagactgaataactacgtatagtactgtagtattactcggagtacgccagaatgtggtgtggtgtcagtctcaccagcaatctggatttgccaagtgtttctattttttaaaaaaaa

SEQ ID NO: 10 - protein, 367 aa, Zea mays
MGSLASERKVVGWAARDATGHLSPYSYTLRNTGPEDVVVKVLYCGICHTDIHQAKNHLGASKYPMVPGHEVVGEVVEVGPEVAKYGVGDVVGVGVIVGCCRECSPCKANVEQYCNKKIWSYNDVYTDGRPTQGGFASTMVVDQKFVVKIPAGLAPEQAAPLLCAGVTVYSPLKHFGLTNPGLRGGILGLGGVGHMGVKVAKAMGHHVTVISSSSKKRAEAMDHLGADAYLVSSDAAAMAAAADSLDYIIDTVPVHHPLEPYLALLKLDGKLVLLGVIGEPLSFVSPMVMLGRKAITGSFIGSIDETAEVLQFCVDKGLTSQIEVVKMGYVNEALERLERNDVRYRFVVDVAGSNVEAEAAAADAASN

SEQ ID NO: 11 - DNA, 1517 bp, Zea mays
gtgcgggctcgtctccatcgcccgccacccgctccgtcgtcgttcgtccccgccgcgccgatcccgaatcgaatggggagcctggcgtccgagaggaaggtggtcgggtgggccgccagggacgccaccggacacctctccccctactcctacaccctcaggaacacaggccctgaagatgtggtggtgaaggtgctctactgcgggatctgccacacggacatccaccaggcaaagaaccacctcggggcttcaaagtatcctatggtccctgggcacgaggtggtcggcgaggtggtggaggtcgggcccgaggtggccaagtacggcgtcggcgacgtggtaggcgtcggggtgatcgttgggtgctgccgcgagtgcagcccctgcaaggccaacgttgagcagtactgcaacaagaagatctggtcatacaacgacgtctacactgatggacggcccacgcagggtggattcgcctccaccatggtcgtcgaccagaagtttgtggtgaagatcccggcaggtctggctccggagcaagcggcgccgctgctgtgcgctggcgtgacggtgtacagcccgctgaagcactttgggctgacgaccccgggcctccgtggcggcatcctgggcctcggcggcgtgggccacatgggcgtgaaggtagccaaggccatgggccaccacgtgacggtgatcagctcgtcgtccaagaagcgcgcggaggcaatggaccacctcggcgcggacgcgtacctagtgagctcggacgccgcggccatggggccggccgccgactcgctggactacatcatcgacacggtgcccgtgcaccacccgctggagccgtacctggcgctgctgaagctggacggcaagctcgtgctgctgggcgtcatcggcgagcccctgagcttcgtgtcgcccatggtgatgctggggcggaaggccatcacggggagcttcatcggcagcatcgacgagaccgctgaggtgcttcagttctgcgtcgacaaggggctcacctcccagatcgaggtggtcaagatggggtacgtgaacgaggcgctggagcggctggagcgcaacgacgtccgctaccgcttcgtcgtcgacgtcgccggtagcaacgtcgaggcggaggcggcggcggcggatgcggccagcaactgatggcaccgcgtcgtcgagtcgaaccacgtctgtgcgccgcgtgcaacgttcgttcgggtcgagtctgcgtgcaacgttctgcttcctttactagttgttgtctttccgccttcttgccgttctgttctgggctttgagatgagacgatggatggtcagtttttaatgtcagactgaataactacgtatagtactgtagtattactcggagtacgccagaatgtggtgtggtgtcagtctcaccagcaatctggatttgccaagtgtttctattttttcttcggtttgcccgagtgtttgtgattgttaagaactacgttattacggatcgtcaaaaaaaaa

SEQ ID NO: 12 - protein, 367 aa, Zea mays
MGSLASERKVVGWAARDATGHLSPYSYTLRNTGPEDVVVKVLYCGICHTDIHQAKNHLGASKYPMVPGHEVVGEVVEVGPEVAKYGVGDVVGVGVIVGCCRECSPCKANVEQYCNKKIWSYNDVYTDGRPTQGGFASTMVVDQKFVVKIPAGLAPEQAAPLLCAGVTVYSPLKHFGLTTPGLRGGILGLGGVGHMGVKVAKAMGHHVTVISSSSKKRAEAMDHLGADAYLVSSDAAAMGPAADSLDYIIDTVPVHHPLEPYLALLKLDGKLVLLGVIGEPLSFVSPMVMLGRKAITGSFIGSIDETAEVLQFCVDKGLTSQIEVVKMGYVNEALERLERNDVRYRFVVDVAGSNVEAEAAAADAASN

SEQ ID NO: 13 - DNA, 3771 bp, Zea mays
tgcctctggctgcttcgtcttcctccccttccttgtcctctgcctctgctgcactctgctccacccccttttttctttcccccatccatggtgcagcttttgccggcgctggtggcgctcgcccttctcctcgtcagaccggtggcggatgcggcgatggctaaatacacgttcacagtaactctctctctctctctctctctgtgtgtgtgtgtgtgtgtgtgtgtctctcttccatctccatgcagagtcgtatcgtattcgatcagtctcatctggtagaattctccaaactgaagttttccgcaagacgagtagaccaccaacttgtctcactcagtcgcgtttccatgaaagaacaagctgttaggcgcgcgagcgaggtacactccagctagcctagctcatcatgaattcagcgtcaggttagccaaccaaagccggacaaaactggagaaaccgtacgcctagaaaccctgaactcgctgctagtagttgggagggttcagttgctaggatttgataggaacatccttgtctgcaatgcatgcacagcacagcagagcacgggccccctagctcgcgaacgacgccatggcccatggttatctttccttttcgagggacacgcatttttttcctccccgcgtgtttatattaaaacggcatctgtgcttatctgtaatgtgttgctataaattaggtggggagcatgcagatcagtcagctgtgcagcagcaccagcattattgcggtgaacgggcagctgccgggcccgtcgatagaagtgaacgaaggcgacgatgtcgtcgtcaaggtcgtcaacaactcgccgtacaacgtgaccatccactggtaagtggtaactcctactactactactgccacttgagcaaaaaaaaaaaaaaaaagaagaagaagaagaagcagggcagggcaggcctgcatactggtttgctatgagtcgtttgctctctttagacacatcctgtgacatcaaagaaggtgccgtaaggaacctgccatcccagcgttttattggatcactggtgagatggttttgataggtttatctctgcccttccttagcctccacccggaagccgaaagtgaagcctctgattaggacgtcttacctgtcgaaacatagcctgtaactgtatctgtatggatgcccatccaaatattcgccaagtgtttaaaacagagtgaatccaaaaaggagagtgtgccatataaaactccacttaggacactcggtacttcggtagtggcttagcgtcagaatcaccaagaaaggtaacggaggttagtctagactgacagcgccagtaattatgagcaccccggcccgttaataataaagaacgatctgcacgcggtttccacaccgtcttttggacctgaaagctacggcagtggtctctattcggcatggcaccagctccacttgagtctgaaactttgaatctccagtgatggcctaacctgacacttcgggcgcttggcttgctgcgagaattgacaggcatggtgtgctccagctcatgaccccgtgggcggacggcccgagcatggtgacgcagtgccccatccagcccagcagctcctacacgtaccggttcagcgtgccggggcaggagggcacgctgtggtggcacgcgcactcctccttcctccgcgccacggtgtacggcgccttcatcatccgcccgaggcgcggcaacgcctaccctttcccggcgccggacaaggaggtccccatcgtgctcggtaatcgctgccgctcgtgcatagtatctaatagcctgttgtttgttgatcgaggacacaaagaagagtcctgacgttttcttgtctctggatgaggctgtcgtcatgagcaggcgagtggtggaaccgcaatgtagtcgacgtcgagagcgacgccatcttggccggtcagcttcccgcccagtccgacgcgttcacagttaacggcaaaaccggcctgctgtaccagtgcgcgagtacgtgtgcatcgcgtcatcgtcgatcacttttggtgaaactgtaaattgcaggtagtgacagagagattgtgtactatacagacgagacgttcacggcggtggtggagcccagcacgagggtgctcctccgggtcgtcaacgccggcctcaactcgcacctcttcttcaagctggccggccacaacttcaccgtcgtcgccgtggacgcgggctacacctccaacctcaacacggacacgctcgtgctcgcgccgggccagaccgtggacgcgctcgtcaccaccgccgccgcgcccgggagctactacatggcggtgctggcgcacgacaccatgagcccgctcgcgttcgcggcgtcggacacgaccaccgccaccgcgatcctccagtacaacggcacgtcgtccaccaacccgcccgcgatgcccgccatgccgtccagctccgactccgggacggccaacgccttctacttcgggctgaggggcctgggcgcgcccgcggtgccggcgccggtggacgtgagcatgaccatcgagctgggcctcggccagctgccctgcgacccgtcgcagaccaggtgcaacgggaccgcggccgccgccgcgatgaacggcgtctcgttccgcctcccgtcccccgagacgtctctcctcggggcgcacgtcgacggcgtcgcgggggtcttcacggcagatttcccggacggtccgccgccgagcggcacggcgatgtcggtggggaccaagctcaagaagctcagctacaactcggtggtggagatcgtgctgcagaacccggcggccgtcccgacggagaaccacccgatccacctccacggcttcaacttcttcgtgctggcgcaggggatgggtaccttcgccccgggaagcgtggcctacaacctggtggacccggtggcccgcaacaccatcgccgtgcctggcggtggctgggctgtcatacgcttcgtcgccaacaatccaggtaggaatatgacatactcccattcttctttctgtagattcagcctacaaagatacatgggcccaccgttacattttgggcctaacgaaattttggactcaacgcaggcatgtggttctttcactgccacctggacccgcacgtgcctatgggcctgggcatggtgttccaggtggacagcgggacgacgcccggctccacgctccctacgccgccgggggattgggtgggagtatgcgacgcgcagcactacgcggccgcggcggcggtagcagcagcgccggtgccagttccggccccagccccagtcccagcaccaatcctagcgccagcaccagcagaatcgccgttgccacctccgcgcgcggtggaccacaagccgtcgcccaaccttcctcagcgcagggagcacacgggtacctctaattccgctgctggacggagagctaaggggcacctcgcttgtttcttgtgttctgtcctccttttctttcttcttcgtcaacacaaggcctagctcatgggaagctttggcgtgatacccgcgcagaatctgcagttttgttcgttcgtgtaagtaatatgaaattgttttttctttaaatcttttatacacgtgtatatggatttatattctgatgtaattgtgtgaccttttccttctccacgtgggaagttgtgcatagcaaagttcatgtttagggtttattggctctctgtattcatgatagacattgatacaaagtaatatcatacactacggtttgatttg

SEQ ID NO: 14 - protein, 641 aa, Zea mays
MVQLLPALVALALLLVRPVADAAMAKYTFTVGSMQISQLCSSTSIIAVNGQLPGPSIEVNEGDDVVVKVVNNSPYNVTIHWHGVLQLMTPWADGPSMVTQCPIQPSSSYTYRFSVPGQEGTLWWHAHSSFLRATVYGAFIIRPRRGNAYPFPAPDKEVPIVLGEWWNRNVVDVESDAILAGQLPAQSDAFTVNGKTGLLYQCANETFTAVVEPSTRVLLRVVNAGLNSHLFFKLAGHNFTVVAVDAGYTSNLNTDTLVLAPGQTVDALVTTAAAPGSYYMAVLAHDTMSPLAFAASDTTTATAILQYNGTSSTNPPAMPAMPSSSDSGTANAFYFGLRGLGAPAVPAPVDVSMTIELGLGQLPCDPSQTRCNGTAAAAAMNGVSFRLPSPETSLLGAHVDGVAGVFTADFPDGPPPSGTAMSVGTKLKKLSYNSVVEIVLQNPAAVPTENHPIHLHGFNFFVLAQGMGTFAPGSVAYNLVDPVARNTIAVPGGGWAVIRFVANNPGMWFFHCHLDPHVPMGLGMVFQVDSGTTPGSTLPTPPGDWVGVCDAQHYAAAAAVAAAPVPVPAPAPVPAPILAPAPAESPLPPPRAVDHKPSPNLPQRREHTGTSNSAAGRRAKGHLACFLCSVLLFFLLRQHKA

SEQ ID NO: 15 - DNA, 2289 bp, Zea mays
tgcctctggctgcttcgtcttcctccccttccttgtcctctgcctctgctgcactctgctccacccccttttttctttcccccatccatggtgcagcttttgccggcgctggtggcgctcgcccttctcctcgtcagaccggtggcggatgcggcgatggctaaatacacgttcacagtggggagcatgcagatcagtcagctgtgcagcagcaccagcattattgcggtgaacgggcagctgccgggcccgtcgatagaagtgaacgaaggcgacgatgtcgtcgtcaaggtcgtcaacaactcgccgtacaacgtgaccatccactggcatggtgtgctccagctcatgaccccgtgggcggacggcccgagcatggtgacgcagtgccccatccagcccagcagctcctacacgtaccggttcagcgtgccggggcaggagggcacgctgtggtggcacgcgcactcctccttcctccgcgccacggtgtacggcgccttcatcatccgcccgaggcgcggcaacgcctaccctttcccggcgccggacaaggaggtccccatcgtgctcggcgagtggtggaaccgcaatgtagtcgacgtcgagagcgacgccatcttggccggtcagcttcccgcccagtccgacgcgttcacagttaacggcaaaaccggcctgctgtaccagtgcgcgaacgagacgttcacggcggtggtggagcccagcacgagggtgctcctccgggtcgtcaacgccggcctcaactcgcacctcttcttcaagctggccggccacaacttcaccgtcgtcgccgtggacgcgggctacacctccaacctcaacacggacacgctcgtgctcgcgccgggccagaccgtggacgcgctcgtcaccaccgccgccgcgcccgggagctactacatggcggtgctggcgcacgacaccatgagcccgctcgcgttcgcggcgtcggacacgaccaccgccaccgcgatcctccagtacaacggcacgtcgtccaccaacccgcccgcgatgcccgccatgccgtccagctccgactccgggacggccaacgccttctacttcgggctgaggggcctgggcgcgcccgcggtgccggcgccggtggacgtgagcatgaccatcgagctgggcctcggccagctgccctgcgacccgtcgcagaccaggtgcaacgggaccgcggccgccgccgcgatgaacggcgtctcgttccgcctcccgtcccccgagacgtctctcctcggggcgcacgtcgacggcgtcgcgggggtcttcacggcagatttcccggacggtccgccgccgagcggcacggcgatgtcggtggggaccaagctcaagaagctcagctacaactcggtggtggagatcgtgctgcagaacccggcggccgtcccgacggagaaccacccgatccacctccacggcttcaacttcttcgtgctggcgcaggggatgggtaccttcgccccgggaagcgtggcctacaacctggtggacccggtggcccgcaacaccatcgccgtgcctggcggtggctgggctgtcatacgcttcgtcgccaacaatccaggcatgtggttctttcactgccacctggacccgcacgtgcctatgggcctgggcatggtgttccaggtggacagcgggacgacgcccggctccacgctccctacgccgccgggggattgggtgggagtatgcgacgcgcagcactacgcggccgcggcggcggtagcagcagcgccggtgccagttccggccccagccccagtcccagcaccaatcctagcgccagcaccagcagaatcgccgttgccacctccgcgcgcggtggaccacaagccgtcgcccaaccttcctcagcgcagggagcacacgggtacctctaattccgctgctggacggagagctaaggggcacctcgcttgtttcttgtgttctgtcctccttttctttcttcttcgtcaacacaaggcctagctcatgggaagctttggcgtgatacccgcgcagaatctgcagttttgttcgttcgtgtaagtaatatgaaattgttttttctttaaatcttttatacacgtgtatatggatttatattctgatgtaattgtgtgaccttttccttctccacgtgggaagttgtgcatagcaaagttcatgtttagggtttattggctctctgtattcatgatagacattgatacaaagtaatatcatacactacggtttgatttgaaaaaaaaaaaaaaaaaaaaa

SEQ ID NO: 16 - protein, 641 aa, Zea mays; identical to SEQ ID NO: 14

SEQ ID NO: 17 - DNA, 2044 bp, Zea mays
cttgcttcaaagcgcaaacacacaagaagggcggagctgttgtcatcctgacaatgggcgcgcgtcgtggtctccggcgaggccaagccgccgccgccgccttctccgcatgtcccttcctcgccctcgccgtcgtcctcctcgccttgccggagctcgcagccggcgacacccactactacacgttcaacgtgcaaatgaccaacgtgacacggctgtgcgtgactaagagcatcccgacggtgaacggggagttcccggggccgaagctggtcgtgcgggaaggcgaccgcctcgtggtcaaggttcacaaccacatcaactacaatgtctcgttccactggcacggcgtccggcagctgcgcaacgggtgggcggacgggccgtcgtacatcacgcagtgcccgatccagggcgggcagagctacgtgtacgacttcaccgtcacggggcagcgcggcacgctgtggtggcacgcgcacttctcctggctgcgcgtgcacctctacggcccgctcgtcatcctccccaagcgcggcgagggctacccgttcccgcgcccctacaaggaggtgcccatcctcttcggcgaatggttcaacgcggacacggaggccgtcatcaaccaggccctgcaaacaggcgccggcccaaacgtctccgatgcctacaccttcaatgggcttccaggcccgacatataactgctcgtctaaagacacgtacaagctgaaggtgaaagcccgggaaggacgtacatgctcccggcttcatcaactccgccctcaaacgaacgagctcttcttcggcatcgccaaccacacgctcaccgtcgtcgaggcggacgccagctacgtcaagccattcaccgtcagcacgctcgtcatttcaccggggcagaccatgaacgtgctcctcacgacggcccccagccccgcctccccggcctacgccatggcgatcgcgccctacaccaacacgcagggcacgttcgacaacaccaccgccgcggccgtcctcgagtacgccccgacgccgccgcccgtcgccaccaggaacaacaccctgccccccctaccggccctgccgctgtacaacgacaccggcgcggtgtccaacttctcgcgcaacttccgcagcctgaacagcgcgcggtacccggcgcgcgtgccggcggcggtggaccggcacctgctgttcaccgtggggctcggcacggacccgtgcccgtacaccaaccagacgtgccagggccccaacggcaccaagttcgcggcgtccgtcaacaacaactccttcttccgcccccggaccgcgctcctcgaggcgcactaccggcgccgctacgccggcgtgctcctgggcgacttccccacggccccgccgcacccgttcaactacacgggcaccccgcccaacaacacgttcgtgcagcacggcacgcgggtggtgccgctccgcttcaacgcctccgtggagctggtgctgcagggcaccagcatccagggcgccgagagccacccgctgcacctgcacggctacaacttcttcgtggtcggccaagggttcggcaacttcgacccggtgaacgacccgcccgggtacaacctcgccgaccccgtagagcgcaacaccatcagcgtgcccaccgccggctgggtcgccgtccggttcctcgccgacaacccgggcgtgtggctgatgcattgccacttcgacgtgcacttgagctggggcctgtccatggcgtggcttgtcaacgacggcccgctgccgaacgagaagatgttgcccccgccatccgacctcccaaaatgctgatgacgactggtcgtttatcacccgatcgaggggtagatgggcatttaggaaggttctcctgcttcctgcacgtctgcctacttcctttccttacgatgtttggaactatttggtttggactatttaattaccgtgtgccgatttttggcgagtgcttggatttcgcgatcctcgctgaatccccttttgaaacatgttaatctgtatctatgtaaaaaaaaaaa

SEQ ID NO: 18 - protein, 588 aa, Zea mays
MGARRGLRRGQAAAAAFSACPFLALAVVLLALPELAAGDTHYYTFNVQMTNVTRLCVTKSIPTVNGEFPGPKLVVREGDRLVVKVHNHINYNVSFHWHGVRQLRNGWADGPSYITQCPIQGGQSYVYDFTVTGQRGTLWWHAHFSWLRVHLYGPLVILPKRGEGYPFPRPYKEVPILFGEWFNADTEAVINQALQTGAGPNVSDAYTFNGLPGPTYNCSSKDTYKLKVKAREGRTCSRLHQLRPQTNELFFGIANHTLTVVEADASYVKPFTVSTLVISPGQTMNVLLTTAPSPASPAYAMAIAPYTNTQGTFDNTTAAAVLEYAPTPPPVATRNNTLPPLPALPLYNDTGAVSNFSRNFRSLNSARYPARVPAAVDRHLLFTVGLGTDPCPYTNQTCQGPNGTKFAASVNNNSFFRPRTALLEAHYRRRYAGVLLGDFPTAPPHPFNYTGTPPNNTFVQHGTRVVPLRFNASVELVLQGTSIQGAESHPLHLHGYNFFVVGQGFGNFDPVNDPPGYNLADPVERNTISVPTAGWVAVRFLADNPGVWLMHCHFDVHLSWGLSMAWLVNDGPLPNEKMLPPPSDLPKC

SEQ ID NO: 19 - DNA, 2192 bp, Zea mays
aacacacacatcgttcttcttcttcttcctccgtctcttcgatcgcctggccggccgtgtcgtcccccctctactagacctcaagagcaactctggatagctgcttatcggagcctagagctagaggttgaaccagtccatcatcaactagcgtggccggccatggcgatctcctctgctcttccatgctcctccctcctcatggcggctgcccaactgatgctcctcgcctccgtcgtcgtccaagtgcaaggcatcacgaggcactacgacttcaatgtgaccatggcgaacgtgacacggctgtgcgccagcaagagcatcatcacggtgaacgggcagttccccgggcccaagatcgtggcgagggaaggcgaccggctcgtcatccgcgtcaccaaccacgcccagcacaacatctcgctgcactggcacggcatccggcagctgcgcacggggtgggcggacgggccggcgtacatcacgcagtgcccgatccagacggggcagagctacgtgtacaactacaccgtcgtggggcagcgcggcacgctgtggtggcacgcgcacatctcctggctgcgcgccaccgtctacgggcccctcgtcatcctgcccaagctcggcgtcccctacccgttcccggcgccctacaaggaggtccccgtcatcttcggtgagtggtggctggcggacacggaggtggtgatcaagcaggcgcttcagctcggcgctggccccaatgtctctgacgcccacaccatcaacggcctgccagggccgctctacaactgctctgccaaagacacgtacaagctgaaggtgaagcccgggaagacgtacatgctgcgcctcatcaacgcggcgctcaacgacgagctcttcttctccgtcgccaaccactcgctcacggtcgtcgaggtcgacgccgtctacgtcaagcccttcaccgtcgacacgctgctcatcgcgccgggccagaccaccaacgtgctgctcgccgccaagccatcctacccgggcgccaactactacatgtccgccgcgccctactccacggccaggccggccaccttcgacaacaccaccgtcgccggcatcctcgagtacgagctgtaccccgacgcgccccggccctccgcctccgcggggagcttcaacgaggccctgccgctctacagaccgaccctgccgcagctcaacgacaccagcttcgtcggcaacttcacggccaagctccgcagcctcgcgacgccgcggtacccggcggccgtgccgcggacggtggacaggcggttcttcttcgcggtcgggctcggcacgcacccgtgccccgccaacgccacgtgccagggccccaccaacaccacgcagttcgcggcgtccgtcaacaacgtctccttcgtgctccccaccaaggcgctgctgcactcccacttcaccggcctgtccagcggcgtctactcgccggacttccccgtcgcgcccctggcgccgttcaactacacggggacgccgcccaacaacaccaacgtggccagcgggaccaagctcatggtcgtcccgtacggcgccaacgtggagctcgtcatgcagggcaccagcatcctcggcgtcgagagccacccgctgcacctgcacggcttcaacttcttcgtggtcggccaagggtacggcaactacgaccccgtcaacgacccgtccaagttcaacctcgtcgaccccgtcgagcgcaacaccgtcggcgtgccggccggcggatgggtggccatccgcttcctcgccgacaaccccggggtctggttcatgcattgccatttggaggcgcacacaacatggggcctcaggatggcatggttggtgctcgacggcagcctcccgcaccagaagctgctcccgccgccgtcagacttacccaaatgttgattagactcttcctctatctctatcctgccggtcgcttcaaattaaagggaattgtgaattagacaaatgtttgtttgtttttttgtttactttcttcattgccaattgcaattttttcaacttgcattttaactagtccgttccgttcctagctgacctggacttttttgtaatttttttcttccatttgtttgccaccacaaatgtttttgtacactcctctgaaaataaagaatggcgtgacttgcaccagataaaaaaaaaaa

SEQ ID NO: 20 - protein, 587 aa, Zea mays
MAISSALPCSSLLMAAAQLMLLASVVVQVQGITRHYDFNVTMANVTRLCASKSIITVNGQFPGPKIVAREGDRLVIRVTNHAQHNISLHWHGIRQLRTGWADGPAYITQCPIQTGQSYVYNYTVVGQRGTLWWHAHISWLRATVYGPLVILPKLGVPYPFPAPYKEVPVIFGEWWLADTEVVIKQALQLGAGPNVSDAHTINGLPGPLYNCSAKDTYKLKVKPGKTYMLRLINAALNDELFFSVANHSLTVVEVDAVYVKPFTVDTLLIAPGQTTNVLLAAKPSYPGANYYMSAAPYSTARPATFDNTTVAGILEYELYPDAPRPSASAGSFNEALPLYRPTLPQLNDTSFVGNFTAKLRSLATPRYPAAVPRTVDRRFFFAVGLGTHPCPANATCQGPTNTTQFAASVNNVSFVLPTKALLHSHFTGLSSGVYSPDFPVAPLAPFNYTGTPPNNTNVASGTKLMVVPYGANVELVMQGTSILGVESHPLHLHGFNFFVVGQGYGNYDPVNDPSKFNLVDPVERNTVGVPAGGWVAIRFLADNPGVWFMHCHLEAHTTWGLRMAWLVLDGSLPHQKLLPPPSDLPKC

SEQ ID NO: 21 - DNA, 2310 bp, Zea mays
cagcaacgcacggagcgtacgtacattgcagtagctagctatagctggccggccatcccctctcgctcgctgctaaacacgttccagcttgtttgctcagagaaacagcgcgcgcgcgcgcgcgcacacacacacatcatcatcatcgattcatcgtacacaggatcagagagcttaattagttctagctgctgcatgccccttcgacaacgtccgacgatgggcggcggcggcggcggtgtagctaagatgccggcaggccagctctggttattactgctaggcgtgttgttgttagcatttggagtcccagcccaggcctccaggaatactcactacgacttcgttataactgagacgaaggtcacccgactatgccatgagaagaccatcctggccgtgaacgggcagttcccggggccgaccatctacgcgcgcaaggacgacgtggtcatcgtcaacgtgtacaaccagggctacaagaacatcaccctccactggcacggcgtggaccagccgcggaacccgtggtccgatggcccggagtacatcacgcagtgccccatccagcccggcgccaacttcacctacaagatcatcttcaccgaggaggaaggcacgctgtggtggcacgcgcacagcgaattcgaccgcgccaccgtgcacggcgccatcgtcatccaccccaagcgcggcaccgtctacccctaccccaagccgcacaaggagatgcccatcatcctcggcgagtggtggaacgcggacgtggagcagatcctcctcgagtcccagcggaccggcggcgacgtcaacatttcggacgccaacaccatcaacggccagcccggcgacttcgccccgtgctctaaggaggacaccttcaagatgtccgtggagcacggcaagacgtacctgctccgggtcatcaacgcggggctcaccaacgagatgttcttcgccgtcgccgggcaccgcctcacggtggtcggcaccgacggccgctacctcaggccgttcaccgtcgactacatcctcatctcccccggacagaccatgaacatgctcctcgaggccaactgcgccaccgacggctcagccaacagccgctactacatggctgcgaggccgttcttcaccaacacggcagtcaatgtcgacgacaaaaacaccacggccattgtggagtacacggacgcgccaccctccgcgtccgcggggccaccggactcccccgacctgccggccatggacgacatcgccgcggcgacggcgtacacggcgcagctccggtccctggtcaccaaggagcatccgatcgacgtgccgatggaggtggacgagcacatgctcgtgacgatctccgtcaacacgattccctgcgagcccaacaagacgtgcgccggccccggaaacaaccgcctcgccgcgagcctgaacaacgtcagcttcatgaacccgaccatcgacatcctcgacgcctactacgactccatcagcggcgtgtacgagccggacttccccaacaagccgcccttcttcttcaacttcaccgctcccaacccgccacaggacctctggttcacgaagcggggcaccaaggtgaaggtggtggagtacggcaccatcctggaggtggtgttccaggacacggccatcctcggcgccgagagccaccccatgcacctgcacggcttcagcttctacgtggtgggccgaggcttcggtaacttcgacaaggacaaggaccccgccacgtacaacctggtcgacccgccttaccagaacaccgtctccgtgcccacgggcggttgggctgcaatgcgcttccgagcggcaaatcctggtgtgtggtttatgcattgccactttgatcgtcacacggtgtggggcatggacactgtgttcattgtgaaaaatggcaagggcccggacgctcagatgatgccacgtccccctaacatgcccaagtgctgagaaaacaagggcacgagctacgactgctcgggttgcatgcaaggcgctcgatcaaaccagctaatcttagttgattggttgatttaattatttgtggtacatattttaagtagaacggttcttcaaataaaacggccagttgagatgtaattagtgtcatttgtgttcttttctctttttattcatttgattgtaagagaaaaacaaattcattatatttattatttgtgtcggtctactgctagttcaatctccaagtgtaattaaacaatgtatgtcaaatcatgtatctagtgaaaattcaatataaatgcgtgcttcaaaaaaaaaaaaaaa

SEQ ID NO: 22 - protein, 584 aa, Zea mays
MGGGGGGVAKMPAGQLWLLLLGVLLLAFGVPAQASRNTHYDFVITETKVTRLCHEKTILAVNGQFPGPTIYARKDDVVIVNVYNQGYKNITLHWHGVDQPRNPWSDGPEYITQCPIQPGANFTYKIIFTEEEGTLWWHAHSEFDRATVHGAIVIHPKRGTVYPYPKPHKEMPIILGEWWNADVEQILLESQRTGGDVNISDANTINGQPGDFAPCSKEDTFKMSVEHGKTYLLRVINAGLTNEMFFAVAGHRLTVVGTDGRYLRPFTVDYILISPGQTMNMLLEANCATDGSANSRYYMAARPFFTNTAVNVDDKNTTAIVEYTDAPPSASAGPPDSPDLPAMDDIAAATAYTAQLRSLVTKEHPIDVPMEVDEHMLVTISVNTIPCEPNKTCAGPGNNRLAASLNNVSFMNPTIDILDAYYDSISGVYEPDFPNKPPFFFNFTAPNPPQDLWFTKRGTKVKVVEYGTILEVVFQDTAILGAESHPMHLHGFSFYVVGRGFGNFDKDKDPATYNLVDPPYQNTVSVPTGGWAAMRFRAANPGVWFMHCHFDRHTVWGMDTVFIVKNGKGPDAQMMPRPPNMPKC

SEQ ID NO: 23 - DNA, 2188 bp, Zea mays
ctctctctctctctctctctctctctctctctctctctctctcgcttcagttcggttcggacacagctagctagctgatcgatcgagaggcgagagcaagcaatggccgtcggccgtcgtctctcccctgcgtgcctcctcctccttcgtctcaccgtcgccctcgtggtcctcaccgccctgccggagctcgccgctgcccgcacccgccgctacacgttcaatgtgacgatggcaacggtgacgcgcctgtgcgtgacgaagagcgtccccacggtgaacgggcagttcccggggccgaggctcgtcgtgcgcgagggagaccgactcgtggtgcaggtccacaacaacatcaacagcaacgtaacgttccactggcacggcgtccggcagctgcgcagcgggtgggcggacgggccgtcctacatcacgcagtgcccgatccggcccggccagagctacgcctacgacttccgcatcgtggggcagcgcggcacgctctggtggcacgcgcacttctcctggctccgagccacgctctacggcccgctcgtcatcctcccgccgcgcggcgtcccctacccgttcccaaagcccgacagacaagtcaccctcatgctcggtgagtggttcaacgcggacccggaggccgtgatcaagcaggcgctgcagaccggcggggccccgaacgtctccgacgcctacaccttcaacggcctgcctggccccacctacaactgctcctccggcgacgacacgttcaggctgcgggtgaggcccggccggacgtacttgctgcgcctcgtcaacgcggcgctcaacgacgagctcttcttcgcggtggccaaccacacgctgacggtggtgcaggcggacgccagctacgtcaagccgttcgcggccgccacgctggtcatctcgccgggccagaccatggacgtgctcctcaccgcctccgcctccgccgccccgtcgtccgccttcgccatcgccgtcgcgccctacaccaacaccgtcggcacgttcgacaacaccaccgccgtcgccgtcgtcgagtacggcccgcaccagtccgccgcggcgctccggagcctgcccttgccggcgctcccgcggtacaacgacacggccgcggtggccaacttctcggccatgttccggagcctcgcgagcgcgcggtacccggcgcgcgtgccgcggaccgtggaccgcaggttcttcttcaccgtcgggctgggcgcggacccgtgccggagccgcgtcaacggcacgtgccagggccccaacggcaccaggttcgccgcgtccatgaacaacgtgtccttcgccatgcccaggaccacctccctcctgcaggcgcactaccagcgccgctacagcggcgtgctcgcggccaacttccccgccgtgccgcccacgcgcttcgactacaccggcgcgccgcccaacaacacgttcgtcacccacggcacccgggtcgtgccgctcagcttcaacaccaccgtcgaggtggtgctgcaggacaccagcgtcctgggcgccgagagccacccgctgcacctgcacggctacgacttcttcgtcgtcggcacgggcttcggcaactacgacgccaccaacgacaccgccagatacaacctcgtcgacccggtgcagcggaacaccgtcagcgtccccaccgccggctgggtcgccatccgcttcgtcgccgacaaccccggcgtgtggatcatgcattgccacctggacgtgcacctgacctggggtctggcaatggcgtggctcgtcaacgacgggccgctgcccaaccagaagctgccacctccaccgtccgatatccccaggtgttgatacaatcacgagacattagtctgcagtggttttgatccgtgcacgcgcacatggcggaaccgactcaaatcgattggcctgtgcaacgcgaattttgtgatgatgtctggttttgtcgctgcactgtttcggagacatacaagatccgtaaaagcaagtttattggggtcatgtgtcgttgcgccatttttcttttttttaacgattgtttcatcgcaacttttgtttttttaatgctggacattgttagtgcgtccagtgtgtaaggtatatgttacatatataatgtatcagtcgattggaccagctaatgattcttttttatcaaaaaaaaaaa

SEQ ID NO: 24 - protein, 582 aa, Zea mays
MAVGRRLSPACLLLLRLTVALVVLTALPELAAARTRRYTFNVTMATVTRLCVTKSVPTVNGQFPGPRLVVREGDRLVVQVHNNINSNVTFHWHGVRQLRSGWADGPSYITQCPIRPGQSYAYDFRIVGQRGTLWWHAHFSWLRATLYGPLVILPPRGVPYPFPKPDRQVTLMLGEWFNADPEAVIKQALQTGGAPNVSDAYTFNGLPGPTYNCSSGDDTFRLRVRPGRTYLLRLVNAALNDELFFAVANHTLTVVQADASYVKPFAAATLVISPGQTMDVLLTASASAAPSSAFAIAVAPYTNTVGTFDNTTAVAVVEYGPHQSAAALRSLPLPALPRYNDTAAVANFSAMFRSLASARYPARVPRTVDRRFFFTVGLGADPCRSRVNGTCQGPNGTRFAASMNNVSFAMPRTTSLLQAHYQRRYSGVLAANFPAVPPTRFDYTGAPPNNTFVTHGTRVVPLSFNTTVEVVLQDTSVLGAESHPLHLHGYDFFVVGTGFGNYDATNDTARYNLVDPVQRNTVSVPTAGWVAIRFVADNPGVWIMHCHLDVHLTWGLAMAWLVNDGPLPNQKLPPPPSDIPRC

SEQ ID NO: 25 - DNA, 2488 bp, Zea mays
aggaattcggcacgaggcccactcctccggctcttcttctccaccggcaccagtccaccacctcccacctcctgccaccgcgcggccccaaccaaccacaccgcagcgcagcaatggcgggcaacggcgccatcgtggagagcgacccgctgaactggggcgcggcggcggcggagctggccgggagccacctggacgaggtgaagcgcatggtggcgcaggcccggcagcccgtggtcaagatcgagggctccaccctccgcgtcggccaggtggccgccgtcgcctccgccaaggacgcgtccggcgtcgccgtcgagctcgacgaggaggcccgcccccgcgtcaaggccagcagcgagtggatcctcgactgcatcgcccacggcggcgacatctacggcgtcaccaccggcttcggcggcacctcccaccgccgcaccaaggacgggcccgcgctccaggtcgagctgctcaggcatctcaacgccggaatcttcggcaccggcagcgacgggcacacgctgccgtcggaggtcacccgcgcggcgatgctggtgcgcatcaacaccctcctccagggctactccggcatccgcttcgagatcctcgaggccatcacgaagctgctcaacaccggtgtcagcccctgcctgccgctccggggcaccatcaccgcgtcgggcgacctggtcccgctctcctacatcgccggcctcatcacgggccgccccaacgcgcaggccgtcaccgtcgacggaaggaaggtggacgccgccgaggcgttcaagatcgccggcatcgagggcggcttcttcaagctcaaccccaaggagggcctcgccatcgtcaacggcacgtccgtgggctccgcgctcgcggccaccgtgatgtacgacgccaacgtcctggccgtcctgtcggaggtcctgtccgccgtcttctgcgaggtcatgaacggcaagcccgagtacacggaccacctgacccacaagctgaagcaccacccggggtccatcgaggccgcggccatcatggagcacatcctggatggcagctccttcatgaagcaggccaagaaggtgaacgagctggacccgctgctgaagcccaagcaggacaggtacgcgctccgcacgtcgccgcagtggctgggcccccagatcgaggtcatccgcgccgccaccaagtccatcgagcgcgaggtcaactccgtgaacgacaacccggtcatcgacgtccaccgcggcaaggcgctgcacggcggcaacttccagggcacccccatcggcgtgtccatggacaacgcccgcctcgccatcgccaacatcggcaagctcatgttcgcgcagttctccgagctcgtcaacgagttctacaacaacgggctcacctccaacctggccggcagccgcaaccccagcctggactacggcttcaagggcaccgagatcgccatggcctcctactgctccgagctccagtacctgggcaaccccatcaccaaccacgtgcagagcgcggacgagcacaaccaggacgtgaactccctgggcctcgtctcggccaggaagaccgccgaggcgatcgacatcctgaagctcatgtcgtccacctacatcgtggcgctgtgccaggccgtggacctgcgccacctcgaggagaacatcaaggcgtcggtgaagaacaccgtgacccaggtggccaagaaggtgctgaccatgaacccctcgggcgagctctccagcgcccgcttcagcgagaaggagctgatcagcgccatcgaccgcgaggccgtgttcacgtacgcggaggacgcggccagcgccagcctgccgctgatgcagaagctgcgcgccgtgctggtggaccacgccctcagcagcggcgagcgcggagcgggagccctccgtgttctccaagatcaccaggttcgaggaggagctccgcgcggtgctgccccaggaggtggaggccgcccgcgtggcgtcgccgagggcaccgcccccgtggcgaaccggatcgcggacagccggtcgttcccgctgtaccgcttcgtgcgcgaggagctcggctgcgtgttcctgaccggcgagaggctcaagtcccccggcgaggagtgcaacaaggtgttcgtcggcatcagccagggcaagctcgtggaccccatgctcgagtgcctcaaggagtgggacggcaagccgctgcccatcaacatcaagtaaaagaacgccaaggagaagaggagggaggggaggaaatacgtgaaaaaaaataaaacctgtacacgtgtctgtccagatcgtgccgtcgatcgctcttcgttgttgatttttggttgtattgatcgatccgtatgtgtgcgtgcgtgtgtggcgttgctctttcttcttttgtgtagctgtgccctgccaccggccgacgtgcgtgcatgccatggtggcgggcagcgttttctaaggtcatatactgttgttgtgtttaagta

SEQ ID NO: 26 - protein, 703 aa, Zea mays
MAGNGAIVESDPLNWGAAAAELAGSHLDEVKRMVAQARQPVVKIEGSTLRVGQVAAVASAKDASGVAVELDEEARPRVKASSEWILDCIAHGGDIYGVTTGFGGTSHRRTKDGPALQVELLRHLNAGIFGTGSDGHTLPSEVTRAAMLVRINTLLQGYSGIRFEILEAITKLLNTGVSPCLPLRGTITASGDLVPLSYIAGLITGRPNAQAVTVDGRKVDAAEAFKIAGIEGGFFKLNPKEGLAIVNGTSVGSALAATVMYDANVLAVLSEVLSAVFCEVMNGKPEYTDHLTHKLKHHPGSIEAAAIMEHILDGSSFMKQAKKVNELDPLLKPKQDRYALRTSPQWLGPQIEVIRAATKSIEREVNSVNDNPVIDVHRGKALHGGNFQGTPIGVSMDNARLAIANIGKLMFAQFSELVNEFYNNGLTSNLAGSRNPSLDYGFKGTEIAMASYCSELQYLGNPITNHVQSADEHNQDVNSLGLVSARKTAEAIDILKLMSSTYIVALCQAVDLRHLEENIKASVKNTVTQVAKKVLTMNPSGELSSARFSEKELISAIDREAVFTYAEDAASASLPLMQKLRAVLVDHALSSGERGAGALRVLQDHQVRGGAPRGAAPGGGGRPRGVAEGTAPVANRIADSRSFPLYRFVREELGCVFLTGERLKSPGEECNKVFVGISQGKLVDPMLECLKEWDGKPLPINIK

(19) United States
(12) Patent Application Publication
(10) Pub. No.: US 2008/0235820 A1
(43) Pub. Date: Sep. 25, 2008

(54) Lignin reduction and cellulose increase in crop biomass via genetic engineering

(75) Inventor: Masomeh B. Sticklen, East Lansing, MI (US)

Correspondence Address: Ian C. McLeod;IAN C. McLEOD, P.C., 2190 Commons Parkway, Okemos, MI, 48864, US

(73) Assignee: Board of Trustees of Michigan State University, East Lansing, MI (US)

(21) Appl. No.: 12/077,764

(22) Filed: Mar. 21, 2008

Related U.S. Application Data
(60) Provisional application No. 60/919,693, filed on Mar. 23, 2007.

(51) Int. Cl. A01H 1/02; A01H 5/00; B02B 5/00; C12P 19/00

(52) U.S. Cl. 800/275; 800/320.1; 241/6; 435/72

(57) ABSTRACT

A transgenic maize plant and methods of using the transgenic maize plant having at least a portion of a coding region of one or more lignin biosynthesis pathway enzymes. In one embodiment, the transgenic plant expresses short interfering RNA (siRNA) for the one or more lignin biosynthesis pathway enzymes that forms a double-strand to activate RNA interference (RNAi). The RNAi decreases expression of the one or more lignin biosynthesis pathway enzymes in the transgenic plant. In a second embodiment, the transgenic plant has a cDNA for the one or more lignin biosynthesis pathway enzymes to increase expression of the one or more lignin biosynthesis pathway enzymes in the transgenic plant.

### CROSS-REFERENCE TO RELATED APPLICATION

This application claims benefit to U.S. Provisional Application Ser. No. 60/919,693, filed Mar. 23, 2007, which is incorporated herein by reference in its entirety.

### STATEMENT REGARDING FEDERALLY SPONSORED RESEARCH OR DEVELOPMENT

Not Applicable.

### REFERENCE TO A “NUCLEOTIDE/AMINO ACID SEQUENCE LISTING APPENDIX SUBMITTED ON A COMPACT DISC”

The application contains nucleotide and amino acid sequences which are identified with SEQ ID NOs. A compact disc is provided which contains the Sequence Listings for the sequences. The Sequence Listing on the compact disc is identical to the paper copy of the Sequence Listing provided with the application.

### BACKGROUND OF THE INVENTION

(1) Field of the Invention

The present invention relates to transgenic crop plants. The transgenic plants use RNA interference (RNAi) to reduce lignin content or modify lignin residue configurations of the plants and increase cellulose.

(2) Description of Related Art

Lignocellulosic biomass is the renewable, cheap and available at over 180 million tons per year produced in the United States [1] and 10-50 billion tons per year at global level [2]. In fact, half of the agronomic biomass produced worldwide is rice straw that is burned to waste causing environmental and health problems [3]. Presently, most ethanol produced in the United States is from maize (corn) kernels with a net energy balance [4], mostly because starch by itself is a valuable commodity. The idea that fermentable sugars for alcohol fuels could be produced from crop biomass has been well received by the U.S. Federal government. However, the major economical downsides of biomass refineries include the pretreatment processing of the lignocellulosic matter and the costs of production of microbial cellulases used to convert the cellulose of biomass into fermentable sugars [5]. It is the recent goal of plant genetic engineering to decrease both of these costs and to further increase the cellulose and/or the overall crop biomass yield [6].

After cellulose, lignin is the second most abundant polymer on earth. In the lignocellulosic biomass, crystalline cellulose is embedded in a hemicellulose and lignin matrix causing the need for costly operation of acid and/or heat pretreatment of biomass to remove lignin and hemicellulose and to disrupt the lignocellulosic matter. Tremendous efforts have been exerted towards improvement of methods of pretreatments in order to reduce costs [9]. Decrease in lignin content via manipulation of different lignin biosynthesis pathway genes have been reported [10,11,12]. Dean also reports [12] that down regulation of lignin can accrue without any apparent harm to the plant growth and development. For example, down regulation of Pt4CL1 in transgenic aspen via antisense technology resulted in 45% decrease in lignin with a cocomitant 15% increase in cellulose, doubling the plant cellulose:lignin ratio without any change in lignin composition and without any apparent harm to the plant growth, development and structural integrity. The suppression of Pt4CL1 is reported to be due to a possible change in metabolic flow of hydroxycinnamic acids. It is believed that this effect could be further amplified by multiple gene cotransformation [6]. Basic research is also in progress for a better understanding of lignin biosynthesis pathway [11], so one could reduce lignin without long-term harm to plant growth, development, or defense.

Although lignin modification can decrease lignin content, one must assure that this modification will not result in harm to the non-lignin related molecular components including those associated with plant defense against invading pathogens and insects. In addition, because lignin deposition of specialized plant cells is known to be through a sophisticated spatial and temporal coordination for evolutionary response to the internal and external needs, more basic research is needed to understand the genetic basis of the lignin pathway regulation [23].

U.S. Pat. No. 5,451,514 to Boudet et al., incorporated herein by reference in its entirety, describes the use of sense and antisense RNA to increase or decrease levels of enzyme, such as cinnamyl alcohol dehydrogenase (CAD), in plants for controlling the synthesis of lignin.

U.S. Pat. No. 6,812,377 to Chiang et al. describe the sinapyl alcohol dehydrogenase (SAD) DNA sequence and using the SAD gene for genetically engineering syringyl-enriched lignin plants. U.S. Pat. No. 6,855,864 to Chiang et al. describe the simultaneous transformation of plants with multiple genes, including 4CL, CAld5H, AldOMT, SAD and CAD genes. U.S. Pat. No. 6,969,784 to Chiang et al. describe the down-regulation the p-coumarate Co-enzyme A ligase (CCL) in aspen trees. Each of the above patents to Chiang et al. is incorporated herein by reference in its entirety.

While genetically modified trees with reduced lignin would be useful to improve pulping for the pulp and paper industry, a need remains for improved transgenic crop plants such as maize having reduced or easily deconstructable lignin that can be more readily converted into fermentable sugars to produce ethanol.

### SUMMARY OF THE INVENTION

The present invention provides a transgenic maize plant having at least one DNA comprising: at least one promoter capable of promoting transcription in the transgenic plant; and at least a portion of a coding region of one or more lignin biosynthesis pathway enzymes operably linked to the promoter. In some embodiments, the transgenic plant expresses short interfering RNA (siRNA) for the one or more lignin biosynthesis pathway enzymes that forms a double-strand to activate RNA interference (RNAi) that decreases expression of the one or more lignin biosynthesis pathway enzymes in the transgenic plant. In further embodiments, the DNA is a cDNA, wherein the transgenic plant expresses the cDNA so as to increase expression of the one or more lignin biosynthesis pathway enzymes in the transgenic plant. In further embodiments, the one or more lignin biosynthesis pathway enzymes are selected from the group consisting of PAL, C4H, C3H, COMT, AldOMT, F5H, CAld5H, 4CL, CCR, CCoA-3H, CCoA-OMT, CAD and laccase. In further embodiments, the promoter is a constitutive promoter. In further still embodiments, the promoter is Cauliflower Mosaic Virus 35S Promoter (CaMV 35S). In further still embodiments, the DNA further comprises a translational enhancer. In further embodiments, the translational enhancer is Tobacco Mosaic Virus Q translational enhancer. In further embodiments, the DNA further comprises a polyadenylation signal. In still further embodiments, the polyadenylation signal is nopaline synthase (Nos) polyadenylation signal.

The present invention provides a method for decreasing lignin production or modifying the configuration of lignin in a transgenic maize plant comprising: providing a transgenic maize plant having at least one DNA comprising at least one promoter capable of promoting transcription in the transgenic plant, and at least a portion of a coding region of one or more lignin biosynthesis pathway enzymes operably linked to the promoter; growing the transgenic plant for a time so that the transgenic plant expresses short interfering RNA (siRNA) for the one or more lignin biosynthesis pathway enzymes that form a double-strand and activate RNA interference (RNAi) to decrease expression of the one or more lignin biosynthesis pathway enzymes in the transgenic plant

The present invention provides a method for producing a ground plant material comprising: providing a transgenic maize plant having at least one DNA comprising at least one promoter capable of promoting transcription in the transgenic plant, and at least a portion of a coding region of one or more lignin biosynthesis pathway enzymes operably linked to the promoter; growing the transgenic plant for a time so that the transgenic plant expresses short interfering RNA (siRNA) for the one or more lignin biosynthesis pathway enzymes that form a double-strand and activate RNA interference (RNAi) to decrease expression of the one or more lignin biosynthesis pathway enzymes in the transgenic plant; harvesting the transgenic plant; and grinding the transgenic plant to provide the ground plant material.

The present invention provides a method for converting a transgenic plant to fermentable sugars comprising: providing a transgenic maize plant having at least one DNA comprising at least one promoter capable of promoting transcription in the transgenic plant, and at least a portion of a coding region of one or more lignin biosynthesis pathway enzymes operably linked to the promoter; growing the transgenic plant for a time so that the transgenic plant expresses short interfering RNA (siRNA) for the one or more lignin biosynthesis pathway enzymes that form a double-strand and activate RNA interference (RNAi) to decrease expression of the one or more lignin biosynthesis pathway enzymes in the transgenic plant; harvesting the transgenic plant; grinding the transgenic plant to provide the ground plant material; incubating the ground plant material in one or more cell wall degrading enzymes to produce the fermentable sugars from lignocellulose in the ground plant material; and extracting the fermentable sugars produced from the lignocellulosic material.

### BRIEF DESCRIPTION OF THE DRAWINGS

FIG. 1 is a diagram illustrating the lignin biosynthesis pathway. PAL: phenyl ammonia lyase; C4H: cinnamate 4-hydroxylase; C3H: 4-hydroxycinnamate 3-hydroxylase; OMT: S-adenosyl-methione-caffeate/5 hydroxyferulate-O-methyltransferase; 4CL: hydroxycinnamate-CoA/5-hydroxyferuloyl-Co-A-ligase; CCR: hydroxycinnamoyl-CoA:NADPH oxidoreductase; CCoA-3H: 4-hydroxycinnamoyl-CoA 3-hydroxylase; CCoA-OMT: S-adenosyl-methionine caffeoyl-Co-A/5-hydroxyferuloyl-Co-A-O-methyltransferase; CAD: hydroxycinnamyl alcohol dehydrogenase; Laccase: polymerization peroxidase; glucosyltransferase: udp-Glc: coniferyl alcohol 4-O-glucosyltransferase; glucosidase: coniferrin-specific 4-O-glucosidase (Pathway is adapted from Dean, 2001).

FIG. 2 is a diagram of a plasmid containing any of the lignin biosynthesis pathway enzyme RNAi regulated by the 35S promoter and enhancer. This construct is the same than the one inventors used to produce E1 in corn biomass (U.S. Pat. No. 7,049,485 to Sticklen et al.), with an exception that here the enzyme in kept within the cytoplasm rather than being targeted into the apoplast. Abbreviations: CaMV 35S=Cauliflower Mosaic Virus 35S Promoter; Ω=Tobacco Mosaic Virus Ω translational enhancer; Nos=Polyadenylation signal of nopaline synthase.

FIG. 3 is a diagram of a plasmid containing any of the lignin biosynthesis pathway enzymes regulated by the 35S promoter and enhancer.

FIG. 4 is a diagram of pDM302 construct containing the bar herbicide resistance selectable marker gene controlled by rice actin 1 promoter and Nos terminator. Abbreviations: Act1-5′=rice acting 1 promoter; Hva1=barley Leah Protein coding sequences; PinII-3′=Potato proteinase inhibitor terminator.

### DETAILED DESCRIPTION OF THE INVENTION

All patents, patent applications, government publications, government regulations, and literature references cited in this specification are hereby incorporated herein by reference in their entirety. In case of conflict, the present description, including definitions, will control.

The term “dicot” as used herein refers to all dicotyledonae plants including, but not limited to, tobacco, potato, sugar beet, and all other annual or perennial plants under the dicotyledonae.

The term “monocot” as used herein refers to all monocoltyledonae plants including, but not limited to cereal plants such as maize, rice, wheat, barley, oat, rye, sorghum, millet, and buckwheat. Additionally, monocot plants include sugar cane, switchgrass and other perennial grasses. Other monocots are certain tree species. The transgenic plant of the present invention is a monocot. In some embodiments, the transgenic plant is a monocot selected from the group consisting of maize, rice, wheat, barley, oat, millet, sorghum, sugar cane and a perennial grass.

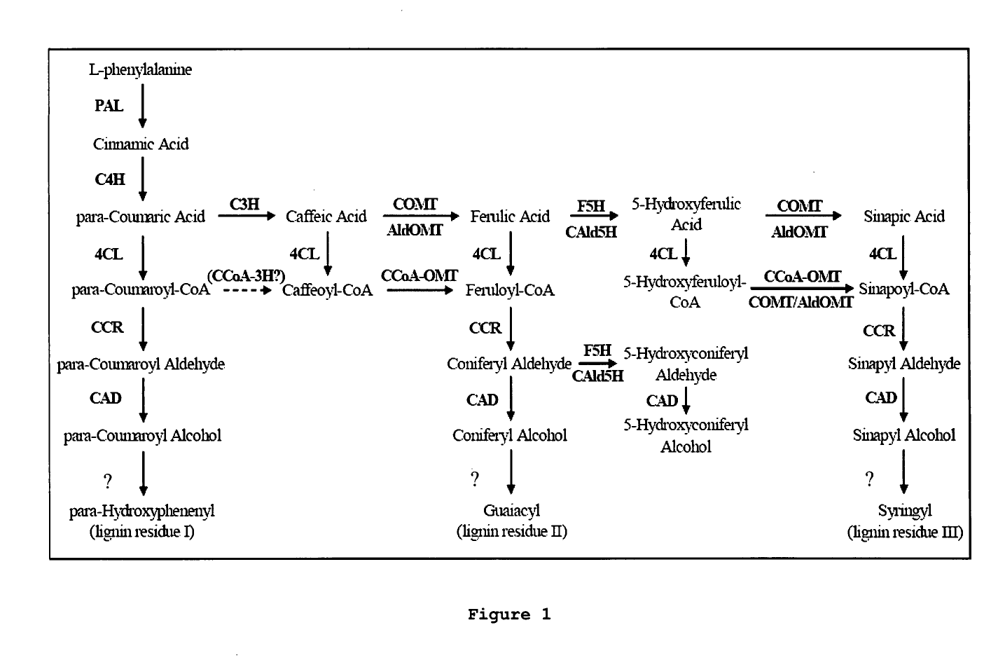

The term “lignin biosynthesis pathway enzymes” as used herein includes, but is not limited to, 4CL and Cald5H. Some examples of lignin biosynthesis pathway enzymes include PAL, C4H, C3H, COMT, AldOMT, F5H, CAld5H, 4CL, CCR, CCoA-3H, CCoA-OMT, CAD and laccase. The diagram of FIG. 1 illustrates where these genes are located in the lignin biosynthesis pathway. FIG. 1 shows the lignin biosynthesis pathway using the following abbreviations: PAL: phenyl ammonia lyase; C4H; cinnamate 4-hydroxylase; C3H: 4-hydroxycinnamate 3-hydroxylase; OMT: S-adenosyl-methione-caffeate/5 hydroxyferulate-O-methyltransferase; 4CL: hydroxycinnamate-CoA/5-hydroxyferuloyl-Co-A-ligase; CCR: hydroxycinnamoyl-CoA: NADPH oxidoreductase; CCoA-3H: 4-hydroxycinnamoyl-CoA 3-hydroxylase; CCoA-OMT: S-adenosyl-methionine caffeoyl-Co-A/5-hydroxyferuloyl-Co-A-O-methyltransferase; CAD: hydroxycinnamyl alcohol dehydrogenase; Laccase: polymerization peroxidase; glucosyltransferase: UDP-Glc: coniferyl alcohol 4-O-glucosyltransferase; glucosidase: coniferrin-specific 4-O-glucosidase.

The term “PAL” or phenylalanine ammonia-lyase as used herein refers to any PAL such as, but not limited to maize PAL. Some examples are set forth as SEQ ID NO: 25-26.

The term “4CL” or “4-coumarate coenzyme A ligase” as used herein refers to any PAL such as, but not limited to maize 4CL. Some examples are set forth as SEQ ID NO: 1-2.

The term “CCR” or “cinnamoyl-CoA reductase” as used herein refers to any CCR such as, but not limited to maize CCR and CCR2. Some examples are set forth as SEQ ID NO: 3-8.

The term “CAD” or “cinnamoyl alcohol dehydrogenase” as used herein refers to any PAL such as, but not limited to maize CAD. Some examples are set forth is SEQ ID NO: 9-12.

The term “laccase” as used herein refers to any laccase such as, but not limited to maize laccase DNA, RNA or proteins having any of the sequences of SEQ ID NO: 13-24. Laccases of any genotype of maize are included such as, but not limited to laccases (Lac1) of GenBank Accession Nos. AY464051, AY464050, AY464049, AY464048, AY464047, AY464046, AY464045, AY464044, AY464043, AY464042, AY464041, AY464040, AY464039, AY464038, AY464037, AY464036, AY464035, AY464034, AY464033, AY464032, AY464031, AY464030, AY464029, AY464028, AY464027, AY464026, AY464025, AY464024, AY464023, AY464022, AY464021, AY464020, AY464019, AY464018, AY464017, and AY464016.

Presently, most ethanol produced in the United States is derived from corn kernel, subsidized with a net energy balance. Plant lignocellulosic biomass is renewable, cheap and globally available at 10-50 billion tons per year. Presently, plant biomass is converted to fermentable sugars for biofuels using pretreatment processes which disrupt the lignocellulose and remove the lignin to allow the access of microbial enzymes for cellulose deconstruction. Both the pretreatments and the production of enzymes in microbial tanks are expensive. Plant genetic engineering can reduce biomass conversion costs by developing crop varieties that (1) have less lignin, (2) are self-producing these enzymes, and (3) have increased cellulose or an overall biomass yield.

Lignocellulosic biomass is composed of crystalline cellulose embedded in a hemicellulose and lignin matrix. The pretreatment methods are presently used to disrupt the lignocellulosic matter, and to mostly remove the lignin to allow the access of cellulose to cellulases. Plant genetic engineering can decrease lignin and/or change the composition of lignin for less need of expensive and harsh pretreatments. Plant genetic engineering can also produce microbial ligninases within the biomass crops, so the lignin content of biomass could be deconstructed during or before bioprocessing. There are three different groups of cellulases working in concert to convert cellulose into glucose. These enzymes include endoglucanase, exoglucanase and the β-glucosidase. Plant genetic engineering has been successfully used to produce these enzymes in plants. Transgenic plants capable of expressing one or more cell wall degrading enzymes are described in U.S. patent application Ser. No. 11/100,270 filed Apr. 6, 2005; Ser. No. 11/489,234 filed Jul. 19, 2006; Ser. No. 11/354,310 filed Feb. 14, 2006; and Ser. No. 09/981,900, filed Oct. 18, 2001 (now U.S. Pat. No. 7,049,485) to Sticklen et al., each of which are hereby incorporated herein by reference in their entirety. The applications describe various DNA constructs that can be used to express heterologous proteins in transgenic plants.

Lignin is a complex phenolics polymer that mostly results from the mixture of para-hydroxyphenyl, guaiacyl and syringyl residues (FIG. 1). Each of these residues results from separate but interconnected pathways. There are two unrelated shorter pathways, one producing caffeoyl CoA and the other producing 5-hydroxyferuloyl CoA or the interactive intermediate which makes 5-hydroxyconiferaldehyde. Manipulation of each of the interconnected pathways of FIG. 1 is expected to modify plant lignin (Sticklen, 2006a; Ragauskas et al., 2006). Maize is the major crop of the U.S. with a DOE goal of commercially using its biomass for conversion into biofuels. At present, the operation costs of chemical pretreatment of feedstock biomass used for removing of lignin to allow the access of cellulase enzymes to the cellulose of biomass is about $1.15 to $2.25/gallon of ethanol (Eggeman, 2005). These costs do not include the production of hydrolytic enzymes, fermentation of sugars into alcohol fuel; or feedstock production, transportation and storage. Therefore, lignin is considered the costly blocking agent in conversion of biomass into alcohol fuels (Sticklen, 2006a; Sticklen 2006b).

Among four maize bm mutants, lignin content was reduced 8% to 30% based on the location of the mutated enzyme in lignin biosynthesis pathway (Chabbert et al., 1994). Also, down-regulation of lignin or modification of lignin structure have been reported in several crops, but not for maize, via down regulation of different lignin biosynthesis pathway enzymes (Sticklen, 2006a). Interestingly, down regulation of 4CL in transgenic quaking aspen (Populus tremuloides) resulted in a 45% decrease in lignin with a concomitant 15% increase in cellulose, doubling the plant cellulose to lignin ratio without any change in lignin composition and without any harm to plant growth, development and structural integrity (Hu et al., 1999). Such compensation has occurred because the quantitative or qualitative changes of one cell wall component often results in alteration of other cell wall components (Boudet et al., 2003). In corn, a decrease in lignin would reduce the costs of pretreatment processes, and an increase in cellulose would increase the level of fermentable sugars from corn biomass.

The present invention promotes understanding of the role of each of the maize lignin biosynthesis pathway enzymes to reduce the maize biomass lignin or modify its chemical structure at a level which reduces the costs of biomass pretreatment processes, without interfering with the crop biotic defense and/or its structural integrity. The present invention down-regulates and/or up-regulates the enzymes associated with maize lignin biosynthesis pathway. The maize genome is mapped (www.ncbi.nlm.nih.gov/Genbank), and the powerful double-stranded RNA mediated interference (RNAi), invented in 1998 (Tabara et al, 1998) as a reverse genetic tool to suppress endogenous gene expression, has revolutionized the technology platform for applications in reducing the expression of endogenous genes. There are over fifty companies that provide RNAi services. The DNA coding sequences are obtained from GeneBank. All of the RNAi needed and the cDNA sequences associated with each of the maize lignin biosynthesis pathway enzyme are obtained commercially.

Maize-specific gene constructs are developed using the RNAi of each of the above enzymes, and mature transgenic plants are developed as is a routine practice in the Sticklen laboratory (see www.msu.edu/˜stickle1; Ransom et al., 2006; Oraby et al, 2006; Biswas et al., 2006; Zhong et al., 2003; Zhong et al., 1996a; Zhong et al., 1996b).

Analysis of the down and up regulation of maize lignin biosynthesis enzymes: The down- and up-regulation of maize lignin biosynthesis genes in transgenic plants, in comparison with untransformed plants, is confirmed by measuring mRNA transcript levels using two molecular methods (i) Microarrays are used to obtain mRNA transcript level ratios by comparison of mRNA transcript levels from control untransformed and transgenic plants using a traditional two-dye experimental design. ii) Real-time PCR complements and validates this analysis, and also allow assessment of mRNA transcripts at low abundance levels which cannot be accurately measured using microarrays. In addition, the latter method is used to obtain absolute quantification of mRNA transcript levels when applied in combination with the calibration curve method (Hashsham et al., 2003; Tourlousse et al., 2006; Musarrat and Hashsham, 2003, Musarrat et al., 2001; Denef et al., 2004; Denef et al., 2006).

Gene-specific oligonucleotide probes (50 nucleotides in length) are designed using dedicated software for all lignin biosynthesis genes based on gene sequences available in public databases such as GenBank (www.ncbi.nlm.nih.gov/Genbank), and genomic sequences of Zea mays cultivar B37 available at www.sequence.org.

Assessment of up- or down-regulation of mRNA transcript levels is performed using the widely applied two-dye experimental design. Reverse-transcription of mRNA transcripts in conjunction with real-time PCR (RT-PCR) analysis of generated cDNA complements and validates microarray-based assessment of mRNA transcript levels. In addition, this allows assessment mRNA transcripts at low abundance levels (less than 10 mRNA transcript copies per cell) which cannot be accurately measured using microarrays. Relative measures of mRNA transcript levels are obtained by comparative analysis of control and transgenic plants to address up- or down-regulation of transcript levels in transgenic plants. In addition, the latter method is used to obtain absolute quantification of transcript levels when combined with calibration curves (Stedtfeld et al.).

Two approaches are adopted for the assessment of mRNA transcript levels using RT-PCR. In the first approach, up- or down-regulation of mRNA transcripts level are addressed by comparative analysis of the mRNA transcript pool from untransformed and transgenic plants. Different mathematical models are used to perform such a comparative analysis using the ΔΔCt model (with or without corrections for amplification efficiencies) being a widely adopted method. In the second approach, transcript levels are quantified absolutely using the calibration curve method. Calibration curves are prepared using the cDNA targets used to construct the cDNA vectors. This curve is then used as a standard for extrapolating quantitative information for mRNA transcripts of unknown concentrations. Again, as is the case for the microarray experiments, both technical and biological replicates are analyzed to obtain statistically meaningful quantification.

The following examples are intended to promote a further understanding of the present invention.

### EXAMPLE 1

The present invention eliminates or reduces the need for expensive pretreatment processes by reducing the lignin content of maize biomass at a level which maize plant would keep its structural integrity in the field, and would defend itself against insects and pathogens. The present invention includes; (1) using the maize genome sequences to develop cDNA and RNAi for each of the lignin biosynthesis enzymes (FIG. 1), (2) genetically engineering maize with each RNAi and cDNA, and (3) evaluating transgenic plants lignin content via three methods including the transcriptom/microarray studies, near infrared spectrophotometery (NIR), and comparing transgenic plants versus the control untransformed for the need for AFEX pretreatment to convert maize biomass into fermentable sugars.

Lignin contains few constituents (Dean, 2001; Ralph, 2005). By definition, lignin is a complex phenolics polymer that mostly results from the mixture of para-hydroxyphenyl, guaiacyl and syringyl residues (FIG. 1). Each of these residues results from separate but interconnected pathways. There are two unrelated shorter pathways, one producing caffeoyl CoA and the other producing 5-hydroxyferuloyl CoA or the interactive intermediate which makes 5-hydroxyconiferaldehyde as seen in FIG. 1. Manipulation of each of the interconnected pathways can modify plant lignin. Lignin biosynthesis pathways are also associated with other functional and defense responsibilities such as those associated with protecting plants from pathogens and insects (Sticklen, 2006a). Certain crops such as maize, sorghum, pearl millet and Arabidopsis mutants have lower lignin content along with higher digestibility as silage. For example, among four different maize bm mutants (Dean, 2001), lignin content was reduced between 8% and 30% based on the location of the mutated enzyme in the lignin biosynthesis pathway (Chabbert et al., 1994; Rogers and Campbell, 2004).

Studies on down-regulation of lignin or modification of lignin structure have been reported in alfalfa to improve digestibility of this crop by rumen (Hans-Joachim, 1998). Other examples are modification of the transgenic tobacco cell wall lignin structure via the use of homologous antisense technology (Blaschke et al., 2004), and the effect of down regulation of C3H on lignin structure, which predictably increased the proportion of para-hydroxyphenyl units relative to normally dominant guaiacyl to syringyl (G:S) ratio (Campbell and Sederoff, 1998; Ralph et al., 2006). Furthermore, the down regulation of CCR (FIG. 1) in populus resulted in more digestible cellulose via Clostridium cellulolyticum and twice the sugar production (Dean, 2001). The down regulation of PAL, which is the master key enzyme responsible for the downstream regulation of the whole lignin biosynthesis flux (FIG. 1), will depend on the level of its suppression (Ragauskar et al., 2006). For example, lignin was completely undetectable when PAL was reduced via anti-sense technology by 15 fold compared to the control untransformed plants (Dean, 2001). Also, it is believed that the overall down regulation of lignin could be further amplified by down regulation of multiple pathway gene co-transformations (Ragauskar et al., 2006).

Maize is the major crop in the U.S., and its biomass is mostly unused to waste. There are over 100 corn grain ethanol plants around the U.S., and there are plans to establish biomass ethanol conversion plants, should the operation costs of biomass conversion be drastically reduced. One method of reducing costs would be to reduce the lignin level or structure so there would be less needs for expensive pretreatment processes. The present invention encompasses both the down regulation and up regulation of each enzyme present in maize lignin biosynthesis pathway (FIG. 1). The transcription of each down regulated and up regulated enzymes with transcription of enzymes in wild-type untransformed maize is compared. The level of lignin produced in each down regulated and up regulated plants versus the control untransformed is measured, and whether the change in regulation of each enzyme has effects on the needs for pretreatment processes to convert maize stock into fermetable sugars is compared. Genetic transformation of maize via immature embryo-derived and multiple apical meristem primordia bombardment systems and other methods are performed as described in U.S. Pat. Nos. 5,767,368, 5,320,961 and 5,281,529 to Zhong et al.; application Ser. No. 11/100,270 filed Apr. 6, 2005; Ser. No. 11/489,234 filed Jul. 19, 2006; Ser. No. 11/354,310 filed Feb. 14, 2006; and Ser. No. 09/981,900, filed Oct. 18, 2001 (now U.S. Pat. No. 7,049,485) to Sticklen et al., each of which are hereby incorporated herein by reference in their entirety.

The present invention reduces the maize biomass lignin content and/or chemical structures so there is less needs for expensive chemical pretreatment processes involved with conversion of maize biomass into fermentable sugars. This is achieved by: 1. Developing two sets of maize-specific plasmid vectors, one for down regulating and the second for up regulating of each of the maize lignin biosynthesis enzymes; 2. Developing transgenic plants using the above two sets of vectors, and confirming each transgene integration and expression in maize plants; and 3. Comparing the down- and up-regulation of lignin biosynthesis in leaves of transgenic plants expressing each of the above transgenes with the control non-transgenic plants using three different techniques including; (a) microarray, (b) INR, and (c) biomass-to-fermentable sugars conversion.

### Methods:

Develop two sets of maize-specific plasmid vectors, one for down regulating and the second for up regulating of maize lignin biosynthesis enzymes: The powerful double-stranded RNA-mediated interference (RNAi) technique, invented in 1998 (Tabara et al, 1998) as a reverse genetic tool to suppress transfected and endogenous gene expression, has revolutionized the technology platform for applications in basic research, target validation and therapeutics. The RNAi technology targets and interferes with the messenger RNA (mRNA), and blocks or down regulates the expression of the gene's protein product. Today, the demand for the use of such technology has resulted in establishment of over fifty RNAi private service sectors with market revenues of over $50 million and a forecasted annual 31.5% growth until 2010 (www.laboratorytalk.com/news/fro/fro185.html). The inventor employs the services of BioRad Laboratories (Hercules, Calif.) that uses a technology which allows the synthesis of small interfering RNAs from DNA templates in vivo for efficient suppression of each of the endogenous lignin biosynthesis enzymes. BioRad Laboratories also produces cDNA for each of the enzymes associated with lignin biosynthesis pathway (FIG. 1).

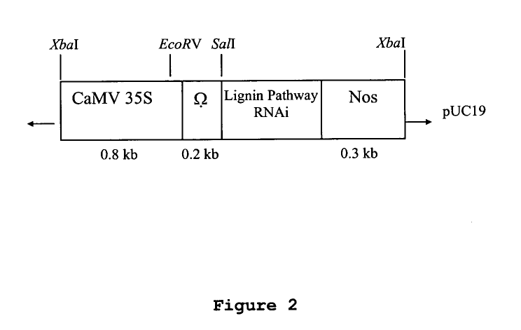

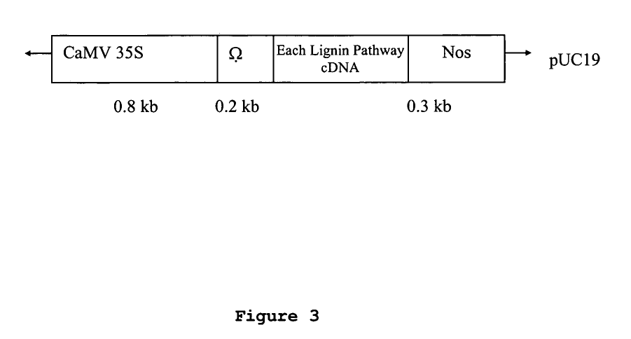

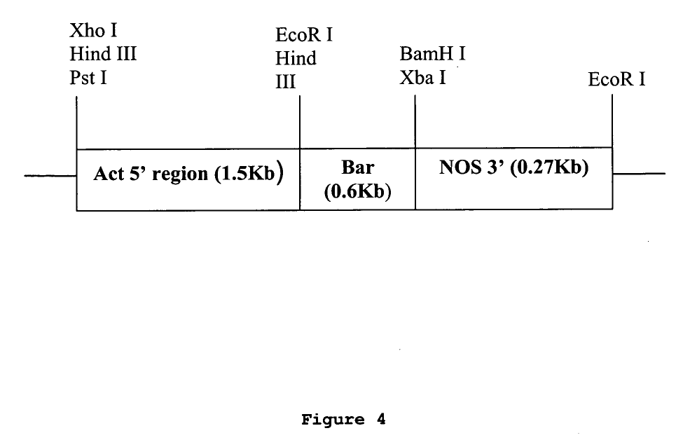

Using the RNAi and cDNA sequences, two sets of maize expression vector constructs (FIG. 2 and FIG. 3) as developed for maize genetic transformation. The first expression vector construct comprises the RNAi of each of the lignin biosynthesis pathway enzymes regulated under a strong constitutive promoter and enhancer as used in inventor Sticklen lab a decade ago (Zhong et al, 1996a, Zhong et al., 1996b). FIG. 2 illustrates a plasmid containing any of the lignin biosynthesis pathway enzyme RNAi regulated by the 35S promoter and enhancer. This construct is the same that one inventor used to produce E1 in corn biomass, with an exception that here the enzyme is kept within the cytoplasm rather than being targeted to the apoplast. CaMV 35S: Cauliflower Mosaic Virus 35S Promoter. Ω: Tobacco Mosaic Virus Ω translational enhancer. Nos: Polyadenylation signal of nopaline synthase. The second set of vectors, as illustrated in FIG. 3, comprise of the full length coding sequences of each of the biosynthesis enzymes shown in FIG. 1 controlled by the same regulatory sequences used in the first set of constructs above (FIG. 2). Each of the constructs in FIG. 2 or FIG. 3 are mixed in ratio of 1:1 with pDM302 (FIG. 4) for maize Biolistic co-bombardment. It is preferred to co-bombard two genes rather than placing the cassette of the gene of interest and the cassette of the selectable marker gene in one construct because the smaller the construct would allow less breakage during Biolistic bombardment.

2. Develop transgenic plants using each set of the above vectors, and confirm transgene integration and expression: Maize plants are grown in greenhouses to maturity. Immature embryos are harvested and cultured in vitro, and immature embryo-derived cell lines are generated and genetically co-bombarded with each of the RNAi constructs (FIG. 2) mixed (1:1 ratio) with the pDM302. The immature embryo-derived cell lines are also genetically co-engineered with each of the lignin biosynthesis enzyme cDNA constructs (FIG. 3) mixed (1:1 ratio) with the pDM302. All cell lines are regenerated into mature maize plants. At least ten different independent transgenic lines will be generated for each of the RNAi and cDNA constructs, and all lines are confirmed for the transgene integration via Southern blotting, and transcription via Northern blotting.

Antibodies are ordered through the Michigan State University Antibody Center using synthetic peptides for each RNAi and each DNA coding sequences of each lignin biosynthesis pathway enzymes.

Western blotting is performed to confirm the translation of each transgene in transgenic maize plants. More details of the Southern, Northern and Western blot analyses are described below.

DNA Isolation and Southern Blot Hybridization Analysis. Confirmation of transgene integration into the plant genome, number of independent transgenic lines, and transgene copy numbers are performed by Southern blot hybridization using each of the transgene coding sequence as a probe. For Southern blots, eight (8) μg of genomic DNA is digested with appropriate restriction enzymes, electrophoresed in 1.0% (w/v) agarose gel, transferred onto Hybond-N+ (Amersham-Pharmacia Biotech) membranes, and fixed with a UV crosslinker (Stratalinker UV Crosslinker 1800, Stratagene, CA) as recommended in the manufacturers'instructions. Each gene-specific probe is generated using PCR amplification of the gene to produce the correct fragment size for each transgene. The amplified fragment is purified using the QIAquick kit (QIAGEN). Probe labeling and detection is obtained using the DIG High Prime DNA Labeling and Detection Starter Kit II (Kit for chemiluminescent detection with CSPD, Roche Co.), following the manufacturer's protocol.

RNA Isolation and Northern Blot Hybridization Analysis. Total RNA samples of untransformed and transgenic plants are isolated from different transgenic lines using the TRI Reagent (Sigma-Aldrich, St. Louis, Mo.) according to the manufacturer's instructions. Also, RNA samples are extracted from untransformed maize and used as a negative control for comparison in this study. Aliquots of RNA (20 μg) are fractionated in 1.2% agarose formaldehyde denaturing gel and blotted on a Hybond-N+ nylon membrane (Amersham Pharmatica Biotech) as specified by the manufacturer. Each specific probe will be generated using PCR amplification of the gene to produce the correct size fragment. The fragment are gel purified using the QIAquick Gel Extraction Kit (QIAGEN Inc., Valencia, Calif.). Probe labeling and transcript detection are obtained using the DIGHigh Prime DNA Labeling and Detection Starter Kit II (Kit for chemiluminescent detection with CSPD, Roche Co.), following the manufacturer's protocol.

Protein Extraction and Western Blot Analysis. Polyclonal antibodies are ordered against each RNAi and coding sequences of each lignin biosynthesis coding sequences through the MSU Antibody Center. Maize total soluble proteins are extracted as described in our reported protocol (Zhong et al., 2003) using the Invitrogen NuPAGE® Bis-Tris Discontinuous Buffer System with the 10% NuPAGE® Novex Bis-Tris Pre-Cast Gel. Total soluble proteins (1 μg), NuPAGE® LDS Sample Buffer (5 μl), NuPAGE® Reducing Agent (2 μl), and deionized water are mixed to a total volume of 20 μl. The samples are heated at 70° C. for 10 minutes prior to electrophoresis using the XCell SureLock™ Mini-Cell with NuPage® MES SDS Running Buffer. The gel are run for about forty-five minutes at 200 V, and then are blotted onto a membrane using the XCell II® Blot Module and NuPAGE® Transfer Buffer at 30 V for one hour, following the manufacturer's protocol. The membrane is placed into blocking buffer (1×PBS, 5% non-fat dry milk, and 0.1% Tween 20) immediately after transfer and incubated at room temperature for one hour with gentle agitation. The antibody is diluted in blocking buffer to a concentration of 1 μg/ml. The blocking buffer is decanted from the membrane, 10 ml of antibody solution is added, and the membrane incubated at room temperature for one hour with gentle agitation. The primary antibody solution is decanted and the membrane washed in washing buffer (1×PBS, 0.1% Tween 20) for 30 minutes with gentle agitation at room temperature, changing the wash solution every five minutes. The enzyme conjugate anti-mouse IgG:HRPO (Transduction Laboratories) is diluted 1:2000 in blocking solution and added to the membrane after decanting the wash buffer. The membrane is incubated with the secondary antibody solution for one hour at room temperature with gentle agitation. Then the antibody solution is decanted from the membrane and the membrane is washed in washing solution as before. For detection, 1 ml each of Stable Peroxide Solution and Luminol/Enhancer Solution (Pierce SuperSignal® West Pico Chemiluminescent Substrate) is mixed and incubated with the membrane for five minutes. The membrane is blotted slightly to remove excess substrate and placed in a plastic envelope. Then, excess liquid and air bubbles are removed. Finally, the blot is exposed to X-ray film (Kodak BioMax XAR Scientific Imaging Film) and developed in a Kodak RP X-OMAT Processor.

Immunofluoresence confocal microscopy of genes translation products. The expression of RNAi and lignin biosynthesis pathway enzyme genes is confirmed using immunofluorescence confocal microscopy. In more details, free-hand sections of fresh leaf tissue from transgenic and untransformed rice plants were isolated and hydrated in NaCl/Pi buffer (0.8% NaCl, 0.02% KCl, 0.14% Na2HPO4.2H2O, and 0.02% KH2PO4 in water) containing 0.5% BSA (BSA/NaCl/Pi) for two minutes. Sections were incubated in primary antibody (rabbit anti-mouse IgG) raised against the E1 enzyme diluted 1:250 in the same buffer, in a moist chamber for three hours. The primary antibody was rinsed off with the BSA/NaCl/Pi buffer and sections were incubated for two hours at room temperature with fluorescein isothiocyanate (FITC)-conjugated secondary antibody (goat anti-(rabbit whole molecule IgG)) diluted 1:250 in the same buffer using same moist chamber. The secondary antibody was then rinsed off with the same buffer. Intracellular localization of the FITC-labeled protein was observed and images were taken using a confocal laser scanning microscopy Zeiss LSM 5 Pascal (Carl Zeiss, Jena, Germany). FITC fluorescence and chloroplast autofluorescence was excited with an argon ion laser, λex=488 nm. Fluorescence emission was detected through a Band Pass (BP) filter, λem=530/30 nm for the FITC (images represented in green) and Long Pass (LP) filter, λem=650 nm for the chloroplast (images represented in red). Either a 63× Plan-apochromat or a 20× Plan-neofluar objective lens was used.

3. Compare possible down regulation and up regulation of lignin biosynthesis in leaves and stems of transgenic plants expressing each of the above transgenes with the control non-transgenic plants using three different techniques including; (a) microarray, (b) NIR, and (c) biomass-to-fermetable sugars conversion.

Microarray technology with 190,000 probe capacity is known in the art (Denef et al., 2003, 2004, 2005a, 2005b, Musarrat and Hashsham, 2003, Musarrat et al., 2001, Wick et al., 2005; Gao et al., 2001, Komolpis, et al., 2002).

Flexibility to change probe design is perhaps the most important characteristic of this technology because it allows alterations to be made to the chip design, simply by providing a new spreadsheet of probe sequences to the in-situ chip synthesizer. This characteristic is critical in most environmental applications of microarrays. When the number of probes are large (e.g., in thousands) and probe design changes frequently, in situ synthesized biochips are the most economical. This technology has been used to develop whole genome arrays for B. xenovorans strain LB400 (Denef et al., 2004), D. hafniense, Ralstonia solanacearum, and environmental detection arrays for community and strain fingerprinting (Hashsham, et al., 2003, Wick, et al., 2005), monitoring waterborne pathogens (Hashsham, et al., 2004), and antibiotic resistance genes (Kruzcewski, et al., 2005).

Statistical design and data analyses: Statistical design of experiments and interpretation of data is an integral part of microarray based experimentation. Its importance takes a whole new meaning for those applications of microarrays that involve mixed microbial communities. Many signals emanating from targets with low abundance are equally important which are currently neglected in pure culture microarray studies. However, reliable measurements of such low abundance signals using microarrays requires enhancements in both technology and data analysis tools. When signals are well above background, traditional triplicate measurements are sufficient. However, when the signals are close to the background, it may be necessary to repeat the measurement more than three times, often up to 20-30 times. Such statistical approaches are incorporated into our experimental design and data analysis (Baushke, et al., 2005). Probabilistic models are synthesized and developed to predict the relationship between marker gene abundance, related environmental factors that affect its transcription and activity, and transformation rate using a Bayesian approach.

The level of lignin in each transgenic versus non-transformed maize using a near infrared spectrophotometer is determined. This device determines the structural makeup and predicts the lignin level in each of the down regulated, and up regulated versus control untranformed plants.

Biomass conversion technology: As described previously (Oraby et al., 2006; Ransom et al., 2006), milled maize stover (about 1 cm in length) down regulated, up regulated and control nontransgenic plants are kept without pretreatment or are pretreated using Ammonia Fiber Explosion technique (AFEX) to examine the level of needs for such pretreatment.

Pretreatment: As described previously (Oraby et al., 2006; Ransom et al., 2006) to perform AFEX pretreatment of the samples, samples of the above maize biomass are transferred to a high pressure Parr reactor with 60% moisture (kg water/kg dry biomass) and liquid ammonia at a ratio of 1.0 (kg of ammonia/kg of dry biomass) is added. As the temperature is slowly raised, the pressure in the vessel increases. The temperature is maintained at 90° C. for five minutes before explosively releasing the pressure. The instantaneous drop of pressure in the vessel occurs causing the ammonia to vaporize, causing an explosive decompression and considerable fiber disruption. The pretreated material is kept under a hood to remove residual ammonia and stored in a freezer until further use.

Enzymatic hydrolysis: As described previously (Oraby et al, 2006; Ransom et al., 2006), the Genencor commercial cellulase enzyme mix (15 FPU/g glucan; 31.3 mg/g glucan) is added to transgenic and control untransformed AFEX-treated and no AFEX-treated grinded maize stover samples. The enzyme hydrolysis is done in a sealed scintillation vial. The substrates are hydrolyzed at a glucan loading of 1% (w:v) in a reaction medium composed of 7.5 ml of 0.1 M, pH 4.8 sodium citrate buffer added to each vial. In addition, 60 μl (600 μg) tetracycline and 45 μl (450 μg) cycloheximide are added to prevent the growth of microorganisms during the hydrolysis reaction. Distilled water is then added to bring the total volume in each vial to 15 ml. All the reactions are done in duplicate to test reproducibility. All hydrolysis reactions are carried out at 50° C. with a shaker speed 90 rpm. About 1 ml of sample is collected at 72 and 168 hours of hydrolysis, filtered using a 0.2 μm syringe filter and kept frozen.

Hydrolyzate are quantified using Waters HPLC by running the sample in Aminex HPX-87P (Biorad) column, against sugar standards. The amount of sugars (hexos and pentose) produced in the enzyme blank and substrate blank are subtracted from the respective hydrolyzate glucose levels. The total sugars produced from the stover of each RNAi, its related lignin biosynthesis enzyme gene, and untransformed plants are compared to confirm the level of down regulated versus the up regulated of lignin in transgenic plants.

A short interfering RNA (SiRNA) is produced for one or more of the lignin biosynthesis pathway enzymes that form a double-strand to activate RNA interference (RNAi) that decreases expansion of the one or more lignin biosynthesis pathway enzymes (SEQ ID NOS: 1 to 26) in the transgenic plant.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

### REFERENCES

### Background of the Invention

1. Kim, S. and Dale, B. E: Global Potential Bioethanol Production from Wasted Crops and Crop Residues. Biomass and Bioenerg 2004, 26, 361-375.
2. Greene, N. (principal author). Growing Energy: How Biofuels Can Help End America's Oil Dependence. Natural Resources Defense Council
3. Kayaba, H, Meguro, H, Muto, H, Kamada, Y, Adachi, T, Yamada, Y, Kanda, A, Yamaguchi, K, Hamada, K et al. Activation of Eosinophils by rice-husk dust exposure: a possible mechanism for the aggravation of asthma during rice harvest. Tohoku J. Exp. Med. 2004, 204, 27-36.
4. Renewable Fuels Association. Homegrown for the homeland: Industry Outlook Report 2005. p. 14.
5. Kabel, M. A., Van der Maarel, M. J. E. C., Klip, G., Voragen, A. G. J. & Schols, H. A. Standard Assays Do Not Predict the Efficiency of Commercial Cellulase Preparations Towards Plant Materials. Biotechnol. Bioeng 2005, 93, 56-63.
6. Ragauskas A J, Williams C K, Davison B H, Britovsek G., Cairney J., Eckert C A, Frederick Jr. W J, Hallett J P, Leak D J, Liotta C L, Mielenz J R, Murphy R, Tempter R, and Tschaplinski T (2006). The path forward for biofuels and biomaterials. Science 2006, 311: 484-489.
7. Sticklen, M. F. Teymouri, S. Maqbool, H. Salehi, C. Ransom, G. Biswas, R. Ahmad and B. Dale. Production of microbial hydrolysis enzymes in biomass crops via genetic engineering. 2nd International Ukrainian Conference on Biomass for Energy 2004, p. 133, 20-22.
8. Oraby H, Venkatesh B, Dale B, Ahmad R, Ransom C and Sticklen M B. Enhanced Conversion of Biomass Polysaccharides into Fermentable Sugars Using Endoglucanase Enzyme Produced in Transgenic Rice. Proceedings of National Academy of Sciences. Submitted (2006).
9. Lynd, L. R., van Zyl, W. H., McBride, J. E. & Laser, M. Consolidated bioprocessing of cellulosic biomass: an update. Curr. Opin. Biotechnol 2005. 16, 577-583.
10. Baudel I will find???
11. Ralph J., Akiyama T., Kim H., Lu F., Schatz P F, Marita J M, Raplph S A, Reddy S., Chen F., and Dixon R A (2006). Effects of Coumarate 3-hydroxylase down-regulation on lignin structure. J. Biol. Chem. 2006. 10.1074/jbc.M511598200.
12. Dean J F D. Synthesis of lignin transgenic and mutant plants. In: Biotechnology of Polymers, From Synthesis to Patents (A. Steinbuchel and Y. Doi). WILEY-VCH Verlag CmbH & Co. KGaA, Weinheim. ISBN: 3-527-31110-6. Vol. ??: Pages ????.
13. Persson S, Wei H., Milne J., Page G P and Somerville C R. Identification of genes required for cellulose synthesis by regression analysis of public microarray data sets. Proc. Natl. Acad. Sci. USA. 2005
14. Anderson-Gunneras S., Mellerrowicz E J., Love J., Segerman B., Ohmiya Y., Coutinho P M., Nilsson P., Henrissat B., Moritz T, and Sundberg B. Biosynthesis of celluloseriched tension wood in populus: global analysis of transcripts and metabolites identifies biochemical and developmental regulations in secondary wall biosynthesis. Plant J. 2006, 45: 144-145.
15. Sakamoto T, Morinaka Y, Ohnishi T, Sunohara H, Fujioka S, Ueguchi-Tanaka M, Mizutani M, Sakata K, Takatsuto S, Yoshida S, Tanaka K, Kitano H, Matsuoka M.
Erect leaves caused by brassinosteroid deficiency increase biomass production and grain yield in rice. Published online: 20 Dec. 2005; |doi:10.1038/nbt1173.
16. Salehi H., Ransom C., Oraby H., and Sticklen M. Delay in flowering and increase in biomass of plants expressing the Arabidopsis floral repressor gene FLC (FLOWERING LOCUS C). Plant Physiol 2005, 162: 711-717.
17. Xiao K, Harrison M, Wang Z. Cloning and Characterization of a Novel Purple Acid Phosphatase Gene (MtPAP1) from Medicago truncatula Barrel Medic. J. Integrative Plant Biol 2006, 48 (2): 208-212.
18. Sinclair T R., Purcell L C, and Sneller C H. Crop transformation and the challenge to increase yield potential. Trends Plant Sci. 2004, 9(2): 70-75.
19. Sahrawy, M., Avila, C., Chueca, A., Canovas, F. M. & Lopez-Gorge, J. Increased sucrose level and altered nitrogen metabolism in Arabidopsis thaliana transgenic plants expressing antisense chloroplastic fructose-1,6-bisphosphatase. J. Exp. Bot 2004. 55, 2495-2503.
20. Teymouri, F., Alizadeh, H., Laureano-Perez; L., Dale, B. E. & Sticklen, M. Effects of Ammonia Fiber Explosion Treatment on Activity of Endoglucanase from Acidothermus cellulolyticus in Transgenic Plant. Appl. Biochem. Biotechnol 2004, 116, 1183-1192.
21. Hood E E. Bioindustrial and biopharmaceutical products from plants. In: New directions for a diverse planet: Proc 4th Intl Crop Sci Congress, Brisbane, Austria, Sep. 26-Oct. 1, 2004. ISBN 1 920842 20 9.
22. Zhong H, Teymouri F, Chapman B, Maqbool S, Sabzikar R, El-Maghraby Y, Dale, B and Sticklen M B. The dicot pea (Pisum sativum L.) rbcS transit peptide directs the Alcaligenes eutrophus polyhydroxybutyrate enzymes into the monocot maize (Zea mays L.) chloroplasts. Plant Sci 2003, 165: 455-462.
23. Rogers L A and Campbell M M. The genetic control of lignin deposition during plant growth and development. New Physiologist. 2004, 164(1): 17-21.
24. Dodd A N., Salathia N., Hall A., Kevei E., Toth R., Nagy F., Hibert J M., Miller A J., and Webb A A R. Plant circadian clocks increase photosynthesis, growth, survival, and competitive advantage. Science 2006, 309: 630-633.
25. NRC Report. Bioconfinement of genetically engineered organisms. The U.S. National Academy of Sciences. Natl. Acad. Sci. Press 2004.
26. Poupin M and Arce-Johnson P. Transgenic trees for a new era. In Vitro Cell Dev. Biol. Plant 2004, 41(2): 91-101.

### Detailed Description of the Invention

Boudet, A. M., S. Kajita, J. Grima-Pettanati, and D. Goffner. 2003. Lignin and lignocellulosics: a better control of synthesis for new and improved uses. Trends Plant Sci. 8: 576-581.
Chabbert B., M. Tollier, B. Monties, Y. Barrier and O. Argillier. 1994. Biological variability in lignification of -expression of the brown midrib maize bm3 mutation in three cultivars. J. Sci. Food. Agric. 64: 349-355.
Denef V. J., Park J, Tsoi T. V., Rouillard J M, Zhang H, Wibbenmeyer J A, Verstraete W, Gulari E, Hashsham S. A., Tiedje J. M. 2004. Biphenyl and benzoate metabolism in a genomic context: outlining genome-wide metabolic networks in Burkholderia xenovorans LB400. Appl Environ Microbiol. 70(8): 4961-70.
Denef V. J., Klappenbach J. A., Patrauchan M. A., Florizone C, Rodrigues J. L., Tsoi T. V., Verstraete W., Eltis L. D., Tiedje J. M. 2006. Genetic and genomic insights into the role of benzoate-catabolic pathway redundancy in Burkholderia xenovorans LB400. Appl Environ Microbiol. 72(1):585-95.
Eggeman, T. and Elander, R. T. 2006. Process and economic analysis of pretreatment technologies. Bioresource Technology 96: 2019-2025.
Gao X., LeProust E. Srivannavit O., Gulari E., Zhou X. 2001. Flexible DNA chip synthesis gated by deprotection using solution photogenerated acids. Nucleic Acids Res. 29, 4744-4750.
Hashsham S. A., Rouillard J. M., Gao X, Callister S, Cole, J. R., Denef, V. J., Tsoi T. V., Wibbenmeyer J, Gulari E, and Tiedje J. M. 2003. Highly Parallel Microbial Detection using In situ Synthesized Flexible Biochips. Proceedings of the 103rd General Meeting of the Am. Society of Microbiology, Washington D.C. May 19-23.
Hu W-J., S. A. Harding, J. Lung, J. L. Popko, J. Ralph, D. D. Stokke, C-J Tsai, and V. L. Chiang. 1999. Repression of lignin biosynthesis promotes cellulose accumulation and growth in transgenic trees. Nature Biotech. 17: 808-812.
Musarrat J, Hashsham S. A. 2003. Customized cDNA microarray for expression profiling of environmentally important genes of Pseudomonas stutzeri strain KC. Teratog Carcinog Mutagen. Suppl 1:283-94.
Musarrat, J., Larabee J., Criddle C., Hashsham S. A. 2001 Monitoring the abundance of mRNA transcripts associated with carbon tetrachloride dechlorination in Pseudomonas stutzeri strain KC under different environmental conditions using DNA microarray. Proceedings of the 101st General Meeting of the American Society for Microbiology. Orlando May 20-24.
Oraby, H., B. Venkatesh, B. Dale, R. Ahmad, C. Ransom, J. Oehmke and M. Sticklen. 2006. Enhanced conversion of plant biomass into glucose using transgenic plant-produced endoglucanase for cellulosic ethanol. Transgenic Res. In press.
Ragauskas A. J., C. K. Williams, B. Davison, G. Britovsek, J. Cairney, C. Eckert, W. Frederick Jr., J. P. Hallett, D. J. Leak, and C. L. Liotta. 2006. The path forward for biofuels and biomaterials. Science 311: 484-489.
Ransom, C., B. Venkatesh, G. Biswas, B. Dale, and M. Sticklen. 2006. Heterologous Acidothermus cellulolyticus 1,4-β-endoglucanase E1 Produced within the maize Biomass Converts Stover into Glucose. Appl. Biochem. Biotechnol. In press.
Stedtfeld R. D., Baushke S. W., Miller S. M, Tiedje J. M., Hashsham S. A. 2006. Microchamber Biochip for Quantitative Detection of Human Pathogens in Environmental Samples. 106th ASM General Meeting, Orlando, May 20-15.
Sticklen, M. 2006a. Plant genetic engineering to improve biomass characterization for biofuels. Curr. Opin. Biotech. 17(3): 315-319.
Sticklen, M. 2006b. Plant biotechnology and genomics. Appl. Biochem. Biotechnol. In press.
Tabara, H., Grishok A., and Mello C. C. 1998. RNAi C. elegans: Soaking in the genome sequence. Science. October 16, 282 (5388): 430-431.
Tourlousse D. M., Miller S. M., Stedtfeld R. D., Herzog A. B., Baushke S. W., Wick L. M., Rouillard J. M., Gulari E., Tiedje J. M., and Hashsham S. A. 2006. Validation of an in situ synthesized oligonucleotide biochip for parallel detection of 12 waterborne pathogens. 106th ASM General Meeting, Orlando, May 20-15.
Zhong H., Teymouri F., Chapman B., Maqbool S., Sabzikar R., El-Maghraby Y., Dale B., and Sticklen M. B. The dicot pea (Pisum sativum L.) rbcS transit peptide directs the Alcaligenes eutrophus polyhydroxybutyrate enzymes into the monocot maize (Zea mays L.) chloroplasts. 2003. Plant Sci. 165: 455-462.
Zhang H., Warkentin D., Sun B., Zhong H., and Sticklen M. B. 1996a. The transmission and expression of two transgenes through outcross and self-cross in maize plants. Theor. Appl. Genet. 92: 752-761.
Zhong H., Zhang S., Sun B., Warkentin D., Wu R., and Sticklen M. B. 1996b. Competence of maize shoot meristem for integrative transformation and inherited expression of transgenes. Plant Physiol. 110: 1097-1107.

### Example

Anderson-Gunneras, S., E. Mellorowicz, J. Love, B. Segerman, Y. Ohmiya, P. Coutinho, P. Nilsson, B. Henrissat, T. Moritz, and B. Sundberg. 2006. Biosynthesis of cellulose-enriched tension wood in populus: global analysis of transcripts and metabolites identifies biochemical and developmental regulators in secondary wall biosynthesis. Plant J., 45: 144-165.
Araki T. 2001. Transition from vegetative to reproductive phase. Curr. Opin. Plant Biol. 4: 63-68.
Arioli T., L. Peng, A. Betzner, J. Burn, W. Wittke, W. Herth, C. Camilleri H. Hofte, J. Planzinski, R. Birch, A. Cork, J. Glover, J. Redmond J, and R. E. Williamson. 1998. Molecular analysis of cellulose biosynthesis in Arabidopsis. Science 279: 717-720.
Altschul, S. F., Madden, T. L., Schäffer, A. A., Zhang, J., Zhang, Z., Miller, W. & Lipman, D. J. 1997. Gapped BLAST and PSI-BLAST: a new generation of protein database search programs. Nucleic Acids Res. 25:3389-3402.
Audic S, Claverie J M. The significance of digital gene expression profiles. Genome Res. 1997 October; 7(10):986-95.
Bailey, M. R. et al. 2004. Improved recovery of active recombinant laccase from maize seed. Appl Microbiol. Biotechnol. 204. 63: 390-397.
Biswas G., C. Ransom, and M. Sticklen. 2006. Expression of biologically active Acidothermus cellulolyticus endoglucanase in transgenic maize. Plant Sci. In press.
Baker, J. O., W. S. Adney, R. A. Nieves, S. R. Thomas, D. B. Wilson, and M. E. Himmel. 1994. A new thermostable endoglucanase, Acidothermus cellulolyticus E1. Synergism with Trichoderma reesei CBH I and comparison to Thermomonospora fusca E5. Appl. Biochem. Biotechnol. 45:245-256.
Qi B., T. Fraser, S. Mugford, G. Dobson, O. Sayanova, J. Butler, J. Napier, A. Stobart and C. Lazarus. 2004. Production of very long chain polyunsaturated omega-3 and omega-6 fatty acids in plants. Bio/technology 22: 739-745.
Blaschke L., M. Legrand, C. Mai and A. Polle. 2004. Lignification and structural biomass production in tobacco with suppressed caffeic/5-hydroxy ferulic acid-O-methyl transferase activity under ambient and elevated CO concentrations. Physiol. Plant. 121: 75-83.
Bohmert K., I. Balbo A. Steinbuchel, G. Tischendorf, and L. Willmitzer. 2002. Constitutive expression of the β-ketothiolase gene in transgenic plants. A major obstacle for obtaining polyhydroxybutyrate-producing plants. Plant Physiol. 128 (4): 1282-1290.
Bothast, R. J. and M. A. Schlicher. 2005. Biotechnological Processes for. Conversion of Corn into Ethanol. Appl. Microbiol. Biotechnol. 67: 19-25.
Boudet, A. M. 2000. Lignins and lignification: Selected issues. Plant Physiol. 38: 81-96.
Boudet, A. M., S. Kajita, J. Grima-Pettanati, and D. Goffner. 2003. Lignin and lignocellulosics: a better control of synthesis for new and improved uses. Trends Plant Sci. 8: 576-581.
Breithaupt, H. 2004. GM plants for your health. EMBO. 5: 1031-1034.
Campbell M. M. and R. R. Sederoff. 1998. Variation in lignin content and composition. Plant Physiol. 110: 3-13
Bateman A, Coin L, Durbin R, Finn R D, Hollich V, Griffiths-Jones S, Khanna A, Marshall M, Moxon S, Sonnhammer E L, Studholme D J, Yeats C, Eddy S R.
Bateman, A., Coin, L., Durbin, R., Finn, R. D., Hollich, V., Griffiths-Jones, S., Khanna, A., Marshall, M., Moxon, S., Sonnhammer, E. L. L., Studholme, D. J., Yeats, C., and Eddy, S. R. 2004. The Pfam protein families database. Nucleic Acids Res. 32:D138-D141.
Baushke, S., S. Kravchenko, L. Wick, R. Stedtfeld, D. Tourlousse, A. Herzog, T. Huynh, J-M. Rouillard, E. Gulari, J. M. Tiedje, and S. A. Hashsham. Statistical Analysis of DNA Microarray Data for Parallel Microbial Detection and Mixed Community Fingerprinting, Abstract submitted to the 105^{th} ASM General Meeting, Atlanta, Ga., May 2005.
Bozdech, Z., Zhu, J., Joachimiak, M. P., Cohen, F. E., Pulliam, B., and DeRisi, J. L. (2003) Expression profiling of the schizont and trophozoite stages of Plasmodium falciparium with a long-oligonucleotide microarray. Genome Biol., 4, R9.
Bruno, W. J., N. D. Socci, and A. L. Halpern. 2000. Weighted Neighbor Joining: A likelihood-based approach to distance-based phylogeny reconstruction, Mol. Biol. Evol. 17:189-197.
Carpita N and McCann M. 2000. The cell wall. In: Biochemistry and Molecular Biology of Plants. Edited by B. Buchanan, W. Gruissem, and R. Jones, American Society of Plant Physiologists. pp. 52-108.
Chabbert B, M. T. Tollier, B. Monties, Y. Barrier and O. Argillier. 1994. Biological variability in lignification of maize-expression of the brown midrib bm3 mutation in three maize cultivars. J. Sci. Food. Agric. 64: 349-355.
Chae, J.-C., B. Song, and G. J. Zylstra. 2004. Identification of the 4-Chlorobenzoate Hydrolytic Dehalogenation Pathway Genes in a Metagenomic Library Derived from a Denitrifying Bacterial Consortium. Abstr. Annu. Meet. Am. Soc. Microbiol.
Chang, H.-K., and G. J. Zylstra. 1998. Novel organization of the genes for phthalate degradation from Burkholderia cepacia DBO1. J. Bacteriol. 180:6529-6537
Chiang C. M., F. S. Yeh, L. F. Huang, T. H. Tseng, M. C. Chung, C. S. Wang, H. S. Lur, J. F. Shaw and S. M. Yu. 2005. Expression of a bifunctional and thermostable amylopullulanase in transgenic rice seeds leads to starch autohydrolysis and altered composition of starch. Mol. Breed. 15: 125-143.
Chou, H. H. and Holmes, M. H. 2001. DNA sequence quality trimming and vector removal. Bioinformatics. 17:1093-1104.
Cigolini, J. F., and G. J. Zylstra. 1999. Molecular cloning of genes for pyrene degradation from Mycobacterium sp. strain PYO1 using universal PCR primers for genes encoding dioxygenase enzymes. Abstr. Am. Soc. Microbiol., Q-180, p. 567.
Cigolini, J., A. K. Goyal, and G. J. Zylstra. 1997. Universal PCR primers for detection, identification, and cloning of genes for dioxygenase enzymes involved in ring oxidation of aromatic compounds. Abstr. Am. Soc. Microbiol., Q 49, p. 463. (student travel award)
Cole, J. R., Chai, B., Farris. R. J., Wang. Q., Kulam, S. A., McGarrell, D. M., Garrity, G. M., and Tiedje, J. M. 2005. The Ribosomal Database Project (RDP-II): sequences and tools for high-throughput rRNA analysis. Nucleic Acids Res. 33:D294-D296.
Courtois S., Cappellano C. M., Ball M., Francou F. X., Normand P., Helynck G., Martinez A., Kolvek S. J., Hopke J., Osburne M. S., August P. R., Nalin R., Guerineau M., Jeannin P., Simonet P., Pernodet J. L. 2003. Recombinant environmental libraries provide access to microbial diversity for drug discovery from natural products. Appl Environ Microbiol. 69:49-55.
Dai, Z., B. S. Hooker, D. B. Anderson, and S. R. Thomas. 2000. Improved plant-based production of E1 endoglucanase using potato: expression optimization and tissue targeting. Mol. Breed. 6: 277-285.
Dean, J. F. D. 2001. Synthesis of lignin in transgenic and mutant plants. In: Biotechnology of Biopolymers, From Synthesis to Patents. Edited by Steinbüchel, A, Doi, Y. Wiley-VCH Verlag, Weinheim, DE. 4-26.
Demain, A. L., M. Newcomb, and J. H. D. Wu. 2005. Cellulase, Clostridia, and Ethanol. Microbiol Mol Biol Rev. 69: 124-154.
Dodd A N, N. Salathia, A. Hall, E. Kevei, R. Toth, F. Nagy, J. M. Hibert, A. J. Miller, A. A. R. Webb. 2005. Plant circadian clocks increase photosynthesis, growth, survival, and competitive advantage. Science 309: 630-633.
Delcher, A. L., Harmon, D., Kasif, S., White, O., and Salzberg, S. L. 1999. Improved microbial gene identification with GLIMMER. Nucleic Acids Res. 27:4636-4641.
Denef V J, Park J, Rodrigues J L, Tsoi T V, Hashsham S A, Tiedje J M. 2003 Validation of a more sensitive method for using spotted oligonucleotide DNA microarrays for functional genomics studies on bacterial communities. Environ Microbiol. 5(10):933-43.
Denef V J, Park J, Tsoi T V, Rouillard J M, Zhang H, Wibbenmeyer J A, Verstraete W, Gulari E, Hashsham S A, Tiedje J M. 2004 Biphenyl and benzoate metabolism in a genomic context: outlining genome-wide metabolic networks in Burkholderia xenovorans LB400. Appl Environ Microbiol. 70(8):4961-70.
Denef, V. J., J. A. Klappenbach, J. L. M. Rodrigues, M. A. Patrauchan, C. Florizone, T. V. Tsoi, W. Verstraete, L. D. Eltis, and J. M. Tiedje, Genetic analysis of the three benzoate catabolic pathways and their associated oxidative stress response in Burkholderia xenovorans LB400. 2005b. Appl. environ. Microbiol. (submitted)
Denef, V. J., M. A. Patrauchan, C. Florizone, J. Park, T. V. Tsoi, W. Verstraete, J. M. Tiedje, and L. D. Eltis. Carbon source and growth phase specific expression of biphenyl, benzoate and C1 metabolic pathways in Burkholderia xenovorans LB400, 2005a J. Bacteriol, (submitted)
Dennis, J. J., and G. J. Zylstra. 1998. Plasposons: modular self-cloning mini-transposon derivatives for the rapid genetic analysis of gram-negative bacterial genomes. Appl. Environ. Microbiol. 64:2710-2715.
Eggeman, T. and Elander, R. T. 2006. Process and economic analysis of pretreatment technologies. Bioresource Technology 96: 2019-2025.
Ewing, B. and P. Green. 1998. Base-calling of automated sequencer traces using Phred. II. Error probabilities. Genome Res. 8:186-194.
Ewing, B., L. Hillier, M. Wendl, and P. Green. 1998. Base-calling of automated sequencer traces using Phred. I. Accuracy assessment. Genome Res. 8:175-185.
Felsenstein, J. 2004. PHYLIP (Phylogeny Inference Package) version 3.6. Distributed by the author. Department of Genome Sciences, University of Washington, Seattle.
Fischer, R., E. Stoger, S. Schillberg, P. Christou, and R. Twyman. 2004. Plant-based production of biopharmaceuticals. Curr. Opin. Plant Biol. 7: 152-158.
Golshan, M., M. Faghihi, T. Roushan-Zamir, M. M. Marandi, B. Esteki, P. Dadvand, M. Farahmand-Far, S. Rahmati, and F. Islami. 2002. Early effects of burning rice farm residues on respiratory symptoms of villagers in suburbs of Isfahan. Int. J. Environ. Health Res. 12: 125-131.
Greene, N., F. E. Celik, B. Dale, M. Jackson, K. Jayawardhana, H. Jin, E. D. Larson, M. Laser, L. Lynd, and D. MacKenzie. 2004. Growing Energy: How Biofuels Can Help End America's Oil Dependence. Natural Resources Defense Council Press. Pp1-86.
Grima-Pettenati J. and D. Goffer. 1999. Lignin genetic engineering revisited. Plant Sci. 145: 51-65.
Gao, X., LeProust, E., Srivannavit, O., Gulari, E., and Zhou, X. 2001. Flexible DNA chip synthesis gated by deprotection using solution photogenerated acids. Nucleic Acids Res. 29, 4744-4750.
Garrity, G. M., J. A. Bell, and T. G. Lilburn. 2004. Taxonomic Outline of the Prokaryotes. Bergey's Manual of Systematic Bacteriology, Second Edition. Release 5.0, May 2004. Springer-Verlag, New York. http://dx.doi.org/10.1007/bergeysoutline200405.
Goyal, A. K., and G. J. Zylstra. 1996. Molecular cloning of novel genes for polycyclic aromatic hydrocarbon degradation from Comamonas testosteroni strain GZ39. Appl. Environ. Microbiol. 62:230-236.
Hashsham S A, Wick L M, Rouillard J M, Gulari E, Tiedje J M. Potential of DNA microarrays for developing parallel detection tools (PDTs) for microorganisms relevant to biodefense and related research needs. Biosensors and Bioelectronics 2004; 20(4):668-683.
Hashsham, S. A., Rouillard, J-M., Gao, X., S. Callister, Cole, J., Denef, V. J., Tsoi, T. V., Wibbenmeyer, J., Gulari, E., and J. Tiedje. 2003. Highly Parallel Microbial Detection using In situ Synthesized Flexible Biochips. Proceedings of the 103rd General Meeting of the American Society of Microbiology, Washington D.C. May 19-23.
Haigler, C. H. 2006. Establishing the cellular and biophysical context of cellulose synthesis. In: The Science and Lore of the Plant Cell Wall: Biosynthesis, Structure and Function, edited by T. Hayashi, Universal Publishers Brown Walker Press. In Press.
Hansen L. D., B. N. Smith, and R. S. 1998. Calorimetry of plant metabolism: A means to rapidly increase agricultural biomass production. Pure & Appl. Chem. 70(3): 687-694.
Henderson I. R., and C. Dean. 2004. Control of Arabidopsis flowering: the chill before the bloom. Development 131: 3829-3838.
Hans-Joachim J. and N. Weiting. 1998. Lignification of plant cell walls: Impact of genetic manipulation. Proc. Natl. Acad Sci. USA. 95: 12742-12743.
Hood E. E., 2004. Bioindustrial and biopharmaceutical products from plants. In: New directions for a diverse planet: Proc 4^{th} Intl Crop Sci Congress, Brisbane, Austria, Sep. 26-Oct. 1, 2004. ISBN 1 920842 20 9.
Horn, M. E., S. L. Woodard, S. L. and J. A. Howard. 2004. Plant molecular farming: Systems and products. Plant Cell Reports 22: 711-720.
Houghton J., J. Ferrel. and S. Weatherwax. 2006. From Biomass to Biofuels: A Roadmap to the Energy Future. In Proceedings of the Biomass to Biofuels Workshop. Office of the Biomass Program of the Office of Energy Efficiency and Renewable Energy, and Office of Biological and Environmental Research Genomics: GTL Program of the Office of Science. Rockville, Md. Dec. 7-9, 2005. PUBLISHER????????????. 186p.
Hu W-J., S. A. Harding, J. Lung, J. L. Popko, J. Ralph, D. D. Stokke, C-J Tsai, and V. L. Chiang. 1999. Repression of lignin biosynthesis promotes cellulose accumulation and growth in transgenic trees. Nature Biotech. 17: 808-812.
Holland, M. J. 2002. Transcript abundance in yeast varies over six orders of magnitude. J. Biol. Chem. 277(17): 14363-14366.
Hughes, T. R., Mao, M., Jones, A. R., Buchard. J., Marton, M. J., Shannon, K. W., Lefkowitz, S. M., Ziman, M., Shelter, J. M., Meyer, M. R. et al. (2001). Expression profiling using microarrays fabricated by an ink-jet oligonucleotide synthesizer. Nat. Biotechnol., 19, 343-347.
Jang H S, Y. P. Lim, Y. Hur. 2003. Expression of flowering related genes in two inbred lines of Chinese cabbage. J Plant Biotechnol 5: 209-214.
Kane, M. D., Jatkoe, T. A., Stumpf, C. R. Lu, J., Thomas, J. D., and Madore, J. M. (2000) Assessment of the specificity and sensitivity of oligonucleotide (50mer) microarrays. Nucleic Acids Res. 28, 4552-4557.
Karp, P. D., S. Paley, and P. Romero. 2002. The pathway tools software. Bioinformatics 18:S1-S8.
Kabel, M. A., M. J. Van der Maarel, E. C., Klip, G., Voragen, and A Schols. 2005. Standard assays do not predict the efficiency of commercial cellulase preparations towards plant materials. Biotechnol. Bioengineering 93:56-63.
Kawagoe Y., and D. P. Delmer. 1997. Pathways and genes involved in cellulose biosynthesis. Genetic Engineering 19: 63-87.
Kayaba H, H. Meguro, H. Muto, Y. Kamada, T. Adachi, Y. Yamada, A. Kanda, K. Yamaguchi, K. Hamada, S. Ueki, and T. Chihara. 2004. Activation of Eosinophils by rice-husk dust exposure: a possible mechanism for the aggravation of asthma during rice harvest. Tohoku J. Exp. Med. 204: 27-36
Kim, J. H., D. Choi and H. Kende. 2003. The AtGRF family of putative transcription factors is involved in leaf and cotyledon growth in Arabidopsis. Plant J. 36: 94-104.
Kim J. H. and H. Kende. 2004. A transcriptional coactivator, AtGIF1, is involved in regulating leaf growth and morphology in Arabidopsis. Proc. Natl. Acad. Sci. USA 101: 13374-13379.
Kim, S, and B. E. Dale. 2004. Global Potential Bioethanol Production from Wasted Crops and Crop Residues. Biomass and Bioenerg. 26: 361-375.
Knauf, M. and M. Moniruzzaman. 2004. Lignocellulosic biomass processing: A perspective. Internat Sugar Jour. 106:147-150
Kim, E. and G. J. Zylstra. 1995. Molecular and biochemical characterization of two meta-cleavage dioxygenases involved in biphenyl and m-xylene degradation by Beijerinckia sp. strain B1. J. Bacteriol. 177:3095-3103.
Komolpis, K., O. Srivannavit, and E. Gulari. 2002. Light-Directed Simultaneous Synthesis of Oligopeptides on Microarray Substrate Using a Photogenerated Acid Biotechnol. Prog., 18, 641-646.
Krajmalnik-Brown, R., T. Holscher, I. N. Thomson, F. M. Saunders, K. M. Ritalahti and F. E. Loffler. 2004. Genetic Identification of a Putative Vinyl Chloride Reductase in Dehalococcoides sp. Strain BAV1. 70: Appl Environ Microb 6347-6351.
Krieger, C. J., P. Zhang, L. A. Mueller, A. Wang, S. Paley, M. Arnaud, J. Pick, S. Y. Rhee, and P. D. Karp. 2004. MetaCyc: a multiorganism database of metabolic pathways and enzymes. Nucl. Acids Res. 32:D438-D442.
Kruszewski, E., Y. Matsumura, V. Denef, L. Wick, K. Yadav, R. Xu, S. Baushke, R. Stedtfeld, J. Tiedje and S. Hashsham. Detection of Vancomycin Resistant Enterococcus faecalis (N00-410) and Enterococcus faecium (N97-330) Using Microarrays, 105^{th} ASM General Meeting, Atlanta, Ga., May 2005.
Liu., H. L., W. S. Li, T. Lei, J. Zheng, Z. Zhang, X. F. Yan, Z. Z. Wang, Y. L. Wang, and L. S. Si. 2005. Expression of Human Papillomavirus type 16 L1 protein in transgenic tobacco plants. Acta Biochim Biophys Sin. 37: 153-158.
Lynd, L. R., W. H. van Zyl, J. E. McBride, and M. Laser. 2005. Consolidated bioprocessing of cellulosic biomass: an update. Curr. Opin. Biotechnol. 16: 577-583
MacDonald, T., G. Yowell, M. McCormack, and M. Bouvier. 2003. Ethanol supply outlook for California. California Energy Commission Report. p. 6.
McCurdy, S. A., T. J. Ferguson, D. F., Goldsmith, J. E. Parker and M. B. Schenker. 1996. Respiratory health of California rice farmers. Am. J. Respir. Crit. Care Med. 153: 1553-1559.
Maroco, J. P., G. E. Edwards, and M. S. and B. Ku. 1999. Photosynthetic acclimation of maize to growth under elevated levels of carbon dioxide. Planta 210: 115-125. Musarrat J., and S. A. Hashsham. Customized cDNA microarray for expression profiling of environmentally important genes of Pseudomonas stutzeri strain KC. Teratogenesis Carcinogenesis and Mutagenesis. 283-294. Suppl. 2003.
Musarrat, J., J. Larabee, C. Criddle, and S. A. Hashsham, 2001. Monitoring the abundance of mRNA transcripts associated with carbon tetrachloride dechlorination in Pseudomonas stutzeri strain KC under different environmental conditions using DNA microarray. Proceedings of the 101st General Meeting of the American Society for Microbiology. Orlando, Fla., May 20-24.
Myers, E. W. (1999) A fast bit-vector algorithm for approximate string matching based on dynamic programming. J. ACM. 46, 539-553.
Nam, J.-M., S. I. Stoeva, C. A. Mirkin, 2004. Bio-Bar-Code-Based DNA Detection with PCR-like Sensitivity, J. Am. Chem. Soc. ASAP article.
Nam, K., W. Rodriguez, and J. J. Kukor. 2001. Enhanced degradation of polycyclic aromatic hydrocarbons by biodegradation combined with a modified Fenton reaction. Chemosphere 45:11-20.
Ni Chadhain, S. M., K. V. Pesce, R. S. Norman, J. J. Kukor, and G. J. Zylstra. 2005. Shifts in Dioxygenase Gene Populations During Polycyclic Aromatic Hydrocarbon Degradation. Abstr. Am. Soc. Microbiol.
Ni Chadhain, S., S. Hicks, J. Schaefer, T. Barkay, and G. J. Zylstra. 2004. Novel Mercuric Reductase Genes Found in Anaerobic Communities of Mercury Contaminated Sediments. Abstr. Annu. Meet. Am. Soc. Microbiol.
Nielsen, H. B., Wernersson, R., and Knudsen, S. (2003) Design of oligonucleotides for microarrays and perspectives for design of multi-transcriptome arrays. Nucleic Acids Res. 31, 3491-3496.
NRC Report. 2004. Bioconfinement of genetically engineered organisms. The U.S. National Academy of Sciences. Natl. Acad. Sci. Press. 265 pp.
NRC Report. 2006. Review of the Department of Energy's Genomics: GTL Program. National Academies Press. 118 pp.
Oliveira, M. E., B. E. Vaughan, and E. J. Rykiel. 2005. Ethanol as fuel: Energy, carbon dioxide balances, and ecological footprint. BioScience. 55:593-602.
Oraby, H., ??????????????????????????. 2006. ???????????????????????????????. Transgenic Res. In press.
Pan, X., C. Arato, N. Gilkes, D. Gregg, W. Mabee, K. Pye, Z. Xiao, X. Zhang, and J. Saddler. 2005. Biorefining of softwoods using ethanol organosolv pulping: preliminary evaluation of process streams for manufacture of fuel-grade ethanol and co-products. Biotechnol. Bioeng. 90: 473-481.
Paul B. G. 2000. Biosynthesis of plant cell wall polysaccharides. Trends in Glycosci. & Glycochem. 12(65): 143-160.
Perlack, R. D., L. L. Wright, A. F. Turhollow, R. L. Graham, B. J. Stokes, and D. C. Erbach. 2005. Biomass as a Feedstock for a Bioenergy and Bioproducts Industry The Technical Feasibility of a Billion-Ton Annual Supply (U.S. Dept. of Energy and U.S. Dept. of Agriculture. 78p. Available at http://feedstockreview.ornl.gov).
Persson S, H. Wei, J. Milne, G. P. Page and C. R. Somerville. 2005. Identification of genes required for cellulose synthesis by regression analysis of public microarray data sets. Proc. Natl. Acad. Sci. USA. 200. 102 (24): 8633-8638.
Pielou, E. C. 1977. Mathematical Ecology. John C. Wiley and Sons, New York.
Ragauskas A. J., C. K. Williams, B. H. Davison, G. Britovsek, J. Cairney, C. A. Eckert, W. J. Frederick Jr., J. P. Hallett, D. J. Leak, and C. L. Liotta. 2006. The path forward for biofuels and biomaterials. Science 311: 484-489.
Ralph J., T. Akiyama, H. Kim, F. Lu, P. F. Schatz, J. M. Marita, S. A. Ralph, S. Reddy, F. Chen, and R. A. Dixon. 2006. Effects of Coumarate 3-hydroxylase down-regulation on lignin structure. J. Biol. Chem. 281(13): 8843-8853.
Ransom, C., B. Venkatesh, G. Biswas, B. Dale, and M. B. Sticklen. 2006. Heterologous Acidothermus cellulolyticus 1,4-β-endoglucanase E1 Produced within the Corn Biomass Converts Corn Stover into Glucose Appl. Biochem. Biotechnol. Accepted for publication.
Reeves P. H., G. Coupland. 2000. Response of plant development to environment: Control of flowering by daylength and temperature. Curr Opin Plant Biol 3: 37-42.
Renewable Fuels Association. 2005. Homegrown for the homeland: Industry Outlook Report. 14p.
Richard R. A. 2000. Selectable traits to increase photosynthesis and yield of crop grains. J. Exp. Bot. 51: 447-458.
Rogers L. A., and M. M. Campbell. 2004. The genetic control of lignin deposition during plant growth and development. New Physiologist 164(1): 17-21. Rodrigues, J. L. M., C. A. Kachel, M. R. Aiello, J. F Quensen, O. V. Maltseva, T. V. Tsoi and J. M Tiedje. 2005 Degradation of products from anaerobic dechlorination of Aroclor 1242 in contaminated sediment using Burkholderia sp. LB400(ohb) and Rhodococcus sp. RHA1(fcb). (Submitted)
Rondon, M. R., August, P. R., Bettermann, A. D., Brady, S. F., Grossman, T. H., Liles, M. R., Loiacono, K. A., Lynch, B. A., MacNeil, I. A., Minor, C., Tiong, C. L., Gilman, M., Osburne, M. S., Clardy, J., Handelsman, J., and Goodman, R. M. 2000. Cloning the soil metagenome: a strategy for accessing the genetic and functional diversity of uncultured microorganisms. Appl. Environ. Microbiol. 66:2541-2547.
Rondon, M., et al. 2000. Cloning the soil metagenome: a strategy for accessing the genetic and functional diversity of uncultured microorganisms. Appl. Environ. Microbiol. 66:2541-2547.
Rouillard, J. M., Zuker, M., and Gulari, E. (2003) OligoArray 2.0: design of oligonucleotide probes for DNA microarrays using thermodynamic approach. Nucleic Acids Res. 31, 3057-3062.
Rutherford, J. Parkhill, J. Crook, T. Horsnell, P. Rice, M-A. Rajandream and B. Barrell. 2000. Artemis: sequence visualisation and annotation. Bioinformatics 16:944-945.
Salehi H, C. Ransom, H. Oraby, and M. Sticklen. 2005. Delay in flowering and increase in biomass of plants expressing the Arabidopsis floral repressor gene FLC (FLOWERING LOCUS C). J. Plant Physiol. 162:711-717.
Samach A., and G. Coupland. 2000. Time measurement and the control of flowering in plants. Bioassays 22: 38-47.
Sticklen, M. et al. 2004. Production of microbial hydrolysis enzymes in biomass crops via genetic engineering. In: Proceedings of the 2nd International Ukrainian Conference on Biomass for Energy. Sep. 20-22, 2004. Kyiv, Ukraine. p 133.
Sticklen, M. 2006a. Plant genetic engineering to improve biomass characterization for biofuels. Curr. Opin. Biotech. 17(3): 315-319.
Sticklen, M. 2006b. Plant biotechnology and genomics: An introduction to Session B of the 28^{th} Symposium on Fuels and Chemicals. Appl. Biochem. Biotechnol. In press.
SantaLuchia, J., Jr. (1998) A unified view of polymer, dumbell, and oligonucleotide DNA nearest-neighbor thermodynamics. Proc. Natl. Acad. Sci. USA 95, 1460-1465.
Saruul P., F. Srien, D. Somers D, and Somac. 2002. Production of biodegradable plastic polymer poly-□-hydroxybutyrarte in transgenic alfalfa. Crop Sci. 42: 919-927.
Sheldon C C, J. E. Burn, P. P. Perez, J. Metzger, Edwards J. A., Peacock W. J., and Dennis E. S. 1999. The FLF MADS box gene: a repressor of flowering in Arabidopsis regulated by vernalization and methylation. Plant Cell 11: 445-458
Simpson G. G., and C. Dean. 2002. Arabidopsis, the rosetta stone of flowering time. Science 296: 285-289.
Sinclair T. R., L. C. Purcell, and C. H. Sneller. 2004. Crop transformation and the challenge to increase yield potential. Trends Plant Sci. 9(2): 70-75.
Singh, S. P., E. Ekanem, T. Wakefield, and S. Corner. 2003. Emerging Importance of Bio-Based Products and Bio-Energy in the U.S. Economy: Information Dissemination and Training of Students. International Food and Agribusiness Management Review 5: 1-15
Singleton, D. R., M. A. Furlong, S. L. Rathburn, and W. B. Whitman. 2001. Quantitative comparisons of 16S rRNA gene sequence libraries from environmental samples. Appl. Environ. Microbiol. 67:4374-4376.
Song, B., J.-C. Chae, S. Ni Chadhain, G. J. Zylstra, and B. Ward. 2004. Identification and Characterization of Genes Encoding Anaerobic Benzoate Degradation in Sediment and Consortium Metagenomic Libraries. Abstr. Annu. Meet. Am. Soc. Microbiol.
Sutton G G; White O; Adams M D; Kerlavage A R. 1995. TIGR Assembler: A New Tool for Assembling Large Shotgun Sequencing Projects. Genome Science & Technology 1:9-19.
Tabara, H., Grishok A., and Mello C C (1998). RNAi C. elegans: Soaking in the genome sequence. Science. October 16, 282 (5388): 430-431.
Tucker, M. P., A. Mohegheghi, K. Grohmann, and M. E. Himmel. 1989. Ultra-thermostable cellulases from Acidothermus cellulolyticus: comparison of temperature optima with previously reported cellulases. Bio/technol. 7: 817-820.
Teymouri, F., H. Alizadeh, L. Laureano-Perez, B. E. Dale, and M. Sticklen. 2004. Effects of ammonia fiber explosion treatment on activity of endoglucanase from Acidothermus cellulolyticus in transgenic plant. Appl. Biochem. Biotechnol. 116: 1183-1192.
Tiedje J M, Tsoi T V, Rodrigues J L M. et al. 2005 Enhancing PCB Bioremediation. Chapter 7 in: Biotreatment of Recacitrnat Compounds. CRC Press (In Press).
Tolstrup, N., Nielson, P., and Kauppinen, S. (2003) OligoDesign: Design of LNA oligonucleotides for gene expression arrays. Nucleic Acids Res. 31, 3758-3762.
Tsoi T V, Plotnikova E G, Cole J R, Guerin W F, Bagdasarian M, Tiedje J M. 1999 Cloning, expression, and nucleotide sequence of the Pseudomonas aeruginosa 142 ohb genes coding for oxygenolytic ortho dehalogenation of halobenzoates. Appl Environ Microbiol. 1999 65(5):2151-62.
Velculescu, V. E., Zhang, L., Zhou, W., Vogelstein, J., Basrai, M. A., Bassett, D. E., Jr., Hieter, P., Vogelstein, B., and Kinzler, K. W., 1997. Cell, 88:243-251.
Wang, X., and Seed, B. (2003) Selection of oligonucleotide probes fro protein coding sequences. Bioinformatics 19, 796-802.
Wick, Lukas M., Thomas S. Whittam, Erdogan Gulari, James M. Tiedje, and Syed A. Hashsham. Novel analysis method of non-equilibrium melting curves from DNA arrays and its application towards specificity assessment of oligonucleotides. (manuscript in preparation)
Wyman C. E., B. E. Dale, T. T. Elander, M. Holtzapple, M. R. Ladisch, and Y. Y. Lee. 2005. Coordinated development of leading biomass technologies. Bioresource Technol. 96: 1959-1966.
Van der Knaap, E., J. H. Kim and H. Kende. 2000. A novel gibberellin-induced gene from rice and its potential regulatory role in stem growth. Plant Physiol. 122: 695-704.
Yabannavar, A., and G. J. Zylstra. 1995. Cloning and characterization of the genes for p-nitrobenzoate degradation from Pseudomonas pickettii strain YH105. Appl. Environ. Microbiol. 61:4284-4290.
Zhong H., F. Teymouri, B. Chapman, S. Maqbool, R. Sabzikar, Y. El-Maghraby, B. Dale, and M. Sticklen. 2003. The dicot pea (Pisum sativum L.) rbcS transit peptide directs the Alcaligenes eutrophus polyhydroxybutyrate enzymes into the monocot maize (Zea mays L.) chloroplasts. Plant Sci. 165: 455-462.
Ziegler, M. T., S. R. Thomas, and K. J. Danna. 2000. Accumulation of a thermostable endo-1,4-b-D-glucanase in the apoplast of Arabidopsis thaliana leaves. Mol. Breed. 6: 37-46.
Ziegelhoffer, T. J., A. Raasch, and S. Austin-Phillips. 2001. Dramatic effects of truncation and subcellular targeting on the accumulation of recombinant microbial cellulase in tobacco. Mol. Breed. 8:147-158.
Zhao, X., Lisa R. Hilliard, Shelly John Mechery, Yanping Wang, Rahul P. Bagwe, Shouguang Jin and Weihong Tan. A rapid bioassay for single bacterial cell quantitation using bioconjugated nanoparticles. 2004. PNAS, vol. 101 (42) 15027-15032.

I claim:

1. A transgenic maize plant having at least one DNA comprising:
(a) at least one promoter capable of promoting transcription in the transgenic plant; and
(b) at least a portion of a coding region of one or more lignin biosynthesis pathway enzymes operably linked to the promoter.

2. The transgenic plant of claim 1, wherein the transgenic plant expresses short interfering RNA (siRNA) for the one or more lignin biosynthesis pathway enzymes that forms a double-strand to activate RNA interference (RNAi) that decreases expression of the one or more lignin biosynthesis pathway enzymes in the transgenic plant.

3. The transgenic plant of claim 1, wherein the DNA is a cDNA, wherein the transgenic plant expresses the cDNA so as to increase expression of the one or more lignin biosynthesis pathway enzymes in the transgenic plant.

4. The transgenic plant of claim 1, 2 or 3, wherein the one or more lignin biosynthesis pathway enzymes are selected from the group consisting of PAL, C4H, C3H, COMT, AldOMT, F5H, CAld5H, 4CL, CCR, CCoA-3H, CCoA-OMT, CAD and laccase.

5. The transgenic plant of claim 1, wherein the promoter is a constitutive promoter.

6. The transgenic plant of claim 5, wherein the promoter is Cauliflower Mosaic Virus 35S Promoter (CaMV 35S).

7. The transgenic plant of claim 1, wherein the DNA further comprises a translational enhancer.

8. The transgenic plant of claim 7, wherein the translational enhancer is Tobacco Mosaic Virus Q translational enhancer.

9. The transgenic plant of claim 1, wherein the DNA further comprises a polyadenylation signal.

10. The transgenic plant of claim 9, wherein the polyadenylation signal is nopaline synthase (Nos) polyadenylation signal.

11. A method for decreasing lignin production or modifying the configuration of lignin in a transgenic maize plant comprising:
(a) providing a transgenic maize plant having at least one DNA comprising at least one promoter capable of promoting transcription in the transgenic plant, and at least a portion of a coding region of one or more lignin biosynthesis pathway enzymes operably linked to the promoter; and
(b) growing the transgenic plant for a time so that the transgenic plant expresses short interfering RNA (siRNA) for the one or more lignin biosynthesis pathway enzymes that form a double-strand and activate RNA interference (RNAi) to decrease expression of the one or more lignin biosynthesis pathway enzymes in the transgenic plant.

12. A method for producing a ground plant material comprising:
(a) providing a transgenic maize plant having at least one DNA comprising at least one promoter capable of promoting transcription in the transgenic plant, and at least a portion of a coding region of one or more lignin biosynthesis pathway enzymes operably linked to the promoter;
(b) growing the transgenic plant for a time so that the transgenic plant expresses short interfering RNA (siRNA) for the one or more lignin biosynthesis pathway enzymes that form a double-strand and activate RNA interference (RNAi) to decrease expression of the one or more lignin biosynthesis pathway enzymes in the transgenic plant;
(c) harvesting the transgenic plant; and
(d) grinding the transgenic plant to provide the ground plant material.

13. A method for converting a transgenic plant to fermentable sugars comprising:
(a) providing a transgenic maize plant having at least one DNA comprising at least one promoter capable of promoting transcription in the transgenic plant, and at least a portion of a coding region of one or more lignin biosynthesis pathway enzymes operably linked to the promoter;
(b) growing the transgenic plant for a time so that the transgenic plant expresses short interfering RNA (siRNA) for the one or more lignin biosynthesis pathway enzymes that form a double-strand and activate RNA interference (RNAi) to decrease expression of the one or more lignin biosynthesis pathway enzymes in the transgenic plant;
(c) harvesting the transgenic plant;
(d) grinding the transgenic plant to provide the ground plant material;
(e) incubating the ground plant material in one or more cell wall degrading enzymes to produce the fermentable sugars from lignocellulose in the ground plant material; and
(f) extracting the fermentable sugars produced from the lignocellulosic material.
